(19) United States
(12) Patent Application Publication
(10) Pub. No.: US 2011/0004124 A1
(43) Pub. Date: Jan. 6, 2011

(54) IMPLANTABLE MEDICAL DEVICE INCLUDING MECHANICAL STRESS SENSOR

(75) Inventors: Joseph F. Lessar, Mora, MN (US); Michael J. Schendel, Andover, MN (US); James D. Neville, Maple Grove, MN (US); Micah A. Litow, Evanston, IL (US)

Correspondence Address: SHUMAKER & SIEFFERT , P.A, 1625 RADIO DRIVE , SUITE 300, WOODBURY, MN, 55125, US

(73) Assignee: Medtronic, Inc., Minneapolis, MN (US)

(21) Appl. No.: 12/767,456

(22) Filed: Apr. 26, 2010

Related U.S. Application Data
(60) Provisional application No. 61/222,265, filed on Jul. 1, 2009.
(60) Provisional application No. 61/291,251, filed on Dec. 30, 2009.

(51) Int. Cl. A61B 5/103

(52) U.S. Cl. 600/587

(57) ABSTRACT

Systems and techniques for determining in vivo mechanical load exerted on an implanted medical device (IMD) are described. The IMD or a device that has a substantially similar form factor as the IMD includes at least one mechanical stress sensor mechanically coupled to a housing or a component within the housing. In some examples, a patient parameter is determined based on a signal indicative of the in vivo mechanical load exerted on the IMD. In addition, in some examples, a processor determines whether a transient or cumulative mechanical load exerted on an IMD exceeded a predetermined threshold. A processor may additionally or alternatively determine whether a pattern in the mechanical loading of the IMD indicates a diversion from a manufacturing, shipping, storage or other handling process.

This application claims the benefit of U.S. Provisional Application No. 61/222,265, which is entitled, “IMPLANTABLE MEDICAL DEVICE INCLUDING MECHANICAL STRESS SENSOR” and was filed on Jul. 1, 2009, and U.S. Provisional Application No. 61/291,251, which is entitled, “IMPLANTABLE MEDICAL DEVICE INCLUDING MECHANICAL STRESS SENSOR” and was filed on Dec. 30, 2009. The entire content of U.S. Provisional Application Nos. 61/222,265 and 61/291,251 is incorporated herein by reference.

### TECHNICAL FIELD

The disclosure relates to medical devices and, more particularly, implantable medical devices.

### BACKGROUND

A variety of implantable medical devices (IMDs) are used for temporary or chronic, e.g., long-term, delivery of therapy to patients suffering from a wide range of conditions, such as conditions related to the cardiac rhythm, chronic pain, tremor, Parkinson's disease, epilepsy, urinary or fecal incontinence, sexual dysfunction, obesity, or gastroparesis. As examples, electrical stimulation generators are used for chronic delivery of electrical stimulation therapies such as cardiac pacing, neurostimulation, muscle stimulation, or the like. Pumps or other therapeutic agent delivery devices may be used for chronic delivery of therapeutic agents, such as drugs. Typically, such IMDs deliver therapy to the patient substantially continuously or periodically according to therapy parameter values defined by a therapy program.

### SUMMARY

This disclosure describes devices, systems, and techniques for determining a mechanical load exerted on an implantable medical device (IMD) that delivers therapy to a patient. The mechanical loads may be internally or externally induced. According to examples described herein, a mechanical stress sensor (e.g., at least one of a strain sensor, a pressure sensor, a force sensor, a load cell, a displacement sensor, and the like) is mechanically coupled to at least one of a housing of an IMD or a component within the housing of the IMD. The sensor generates a signal indicative of a mechanical load exerted on the IMD while the IMD is acutely or chronically (e.g., non-temporarily) implanted within a patient. In addition, in some examples, the sensor generates a signal indicative of a mechanical load exerted on the IMD prior to implantation in a patient (e.g., during shipping, handling or storage) or after explantation from a patient.

In addition, devices, systems, and techniques for determining compressive forces exerted on an IMD by a muscle proximate the implanted device are described. In some examples, a muscle induced compressive force exerted on the implanted medical device is determined based on a transfer function that indicates a relationship between an in-line muscle force (e.g., a force induced by a muscle substantially along a line of action (or contraction) of the muscle) and a force normal to the direction in which the in-line muscle force is induced. The normal force can indicate the compressive forces that a muscle may exert on the IMD when the IMD is implanted under the muscle in a deep direction (e.g., in a submusuclar implant site within a patient).

In one aspect, the disclosure is directed to an implantable medical system comprising a housing, a therapy delivery module substantially enclosed within the housing, wherein the therapy delivery module is configured to deliver therapy to a patient, a component substantially enclosed within the housing, and a mechanical stress sensor mechanically coupled to at least one of the housing or the component. The mechanical stress sensor generates a signal indicative of a mechanical load exerted on the housing or the component. In some examples, the system further comprises a telemetry module, and a processor. The processor is configured to receive the signal from the mechanical stress sensor and transmit information indicative of the signal to an external device via the telemetry module. The housing may be an outer housing of an IMD.

In another aspect, the disclosure is directed to a method comprising implanting a mechanical stress indicator system in a patient, where the mechanical stress indicator system comprises a housing defining a form factor of an implantable medical device that delivers therapy to a patient, a component enclosed within the housing, and a mechanical stress sensor coupled to at least one of the housing or the component, where the mechanical stress sensor generates a signal indicative of a mechanical load exerted on the housing or the component. The housing may be an outer housing of an IMD. The method further comprises, with a processor, determining an in vivo mechanical load exerted on the housing or the component based on the signal generated by the mechanical stress sensor.

In another aspect, the disclosure is directed to an implantable medical system comprising a housing, means for delivering therapy to a patient, wherein the means for delivering therapy is substantially enclosed within the housing, a component substantially enclosed within the housing, and means for generating a signal indicative of a mechanical load exerted on the housing or the component. The housing may be an outer housing of an IMD. In some examples, the system further comprises means for transmitting the signal to an external device.

In another aspect, the disclosure is directed to a method comprising receiving a signal generated by a mechanical stress sensor implanted within a patient, where the signal is indicative of a mechanical load exerted on a housing that defines a form factor of an implantable medical device that delivers therapy to a patient, and where the mechanical stress sensor is coupled to at least one of the housing or a component enclosed within the housing. The method further comprises, with a processor, comparing at least one characteristic of the signal to a predetermined threshold value, and generating an indication based on the comparison.

In another aspect, the disclosure is directed to an implantable medical system comprising a housing defining a form factor of an implantable medical device that delivers therapy to a patient, a component enclosed within the housing, a mechanical stress sensor mechanically coupled to at least one of the housing or the component, wherein mechanical stress sensor generates a signal indicative of a mechanical load exerted on the housing or the component, a telemetry module within the housing, and a processor, which may also be within the housing. The processor is configured to receive the signal from the mechanical stress sensor and transmits information indicative of the signal from the mechanical stress sensor via the telemetry module and automatically determine whether the mechanical load exerted on the housing or the component exceeds a predetermined threshold. In some examples, the implantable medical system includes a therapy delivery module enclosed within the housing. In addition, in some examples, the processor is configured to generate an indication in response to determining the mechanical load exerted on the housing exceeds the predetermined threshold. In some examples, the implantable medical system further comprises a memory, wherein the processor is configured to store mechanical stress information determined based on the signal in the memory. The mechanical stress information may include, for example, at least one of a mean, median, minimum or maximum mechanical load exerted on the housing or a range of mechanical loads exerted on the housing.

In another aspect, the disclosure is directed to an implantable medical system comprising a housing defining a form factor of an implantable medical device that delivers therapy to a patient, a component enclosed within the housing, means for generating a signal indicative of a mechanical load exerted on the housing or the component, and means for automatically determining whether the mechanical load exerted on the housing or the component exceeds a predetermined threshold.

In another aspect, the disclosure is directed to a method comprising, with a processor, determining a parameter of a muscle, determining a first force exerted by the muscle along a first direction, determining a second force exerted by the muscle along a second direction substantially perpendicular to the first direction based on the parameter of the muscle and the first force, and selecting at least one implant parameter for an implantable medical device based on the second force.

In another aspect, the disclosure is directed to a method comprising, with a processor, determining a compressive force exerted by a muscle based on a transfer function that indicates a relationship between the compressive force and an in-line muscle force and at least one muscle parameter, and selecting at least one implant parameter for an implantable medical device based on the compressive force.

In another aspect, the disclosure is directed to a system comprising a user interface, and a processor that is configured to determine a parameter of a muscle, determine a first force exerted by the muscle along a first direction, determine a second force exerted by the muscle along a second direction substantially perpendicular to the first direction based on the parameter of the muscle and the first force, select at least one implant parameter for an implantable medical device based on the second force, and present the at least one implant parameter to a user via the user interface.

In another aspect, the disclosure is directed to a system comprising a memory that stores a transfer function that indicates a relationship between a compressive force exerted by a muscle and an in-line force exerted by the muscle and at least one parameter of the muscle, and a processor that is configured to determine in-line force and at least one parameter of the muscle, and determine the compressive force based on the transfer function and the in-line force and the at least one parameter of the muscle.

In another aspect, the disclosure is directed to a system comprising means for determining a parameter of a muscle, means for determining a first force exerted by the muscle along a first direction, and means for determining a second force exerted by the muscle along a second direction substantially perpendicular to the first direction based on the parameter of the muscle and the first force.

In another aspect, the disclosure is directed to a system comprising means for determining a compressive force exerted by a muscle based on a transfer function that indicates a relationship between the compressive force and an in-line muscle force and at least one muscle parameter, and means for selecting at least one implant parameter for an implantable medical device based on the compressive force.

In another aspect, the disclosure is directed to an article of manufacture comprising a computer-readable storage medium comprising instructions. The instructions cause a programmable processor to determine a parameter of a muscle, determine a first force exerted by the muscle along a first direction, determine a second force exerted by the muscle along a second direction substantially perpendicular to the first direction based on the parameter of the muscle and the first force, and select at least one implant parameter for an implantable medical device based on the second force.

In another aspect, the disclosure is directed to an article of manufacture comprising a computer-readable storage medium comprising instructions. The instructions cause a programmable processor to determine a compressive force exerted by a muscle based on a transfer function that indicates a relationship between the compressive force and an in-line muscle force and at least one muscle parameter, and select at least one implant parameter for an implantable medical device based on the compressive force.

In another aspect, the disclosure is directed to an article of manufacture comprising a computer-readable storage medium comprising instructions. The instructions cause a programmable processor to perform any part of the techniques described in this disclosure. The instructions may be, for example, software instructions, such as those used to define a software or computer program. The computer-readable medium may be a computer-readable storage medium such as a storage device (e.g., a disk drive, or an optical drive), memory (e.g., a Flash memory, random access memory or RAM) or any other type of volatile or non-volatile memory that stores instructions (e.g., in the form of a computer program or other executable) to cause a programmable processor to perform the techniques described in this disclosure.

The details of one or more aspects of the disclosure are set forth in the accompanying drawings and the description below. Other features, objects, and advantages of the examples of the disclosure will be apparent from the description and drawings, and from the claims.

### BRIEF DESCRIPTION OF DRAWINGS

FIG. 1 is a conceptual diagram illustrating an example therapy system in which an implantable medical device (IMD) is configured to deliver electrical stimulation therapy to a heart of a patient.

FIG. 2 is a conceptual diagram illustrating an example therapy system in which an IMD is configured to deliver electrical stimulation therapy to a tissue site proximate a spine of a patient.

FIG. 3 is a conceptual diagram illustrating an example therapy system in which an IMD is configured to deliver a therapeutic agent to a tissue site within a patient.

FIG. 4 is a conceptual functional block diagram of an example IMD.

FIG. 5 is a conceptual functional block diagram of an example IMD that includes a plurality of mechanical load sensors.

FIG. 6 is a conceptual functional block diagram of an example external programmer.

FIG. 7 is a block diagram illustrating an example system that includes an external device, such as a server, and one or more computing devices that are coupled to an IMD and external programmer via a network.

FIG. 8 is a flow diagram of an example technique with which a device may determine whether mechanical load exerted on an IMD is indicative of a mechanical stress acting within the IMD that may affect the operation of IMD.

FIG. 9 is a flow diagram of an example technique for controlling therapy delivery by an IMD to a patient based on a signal generated by a mechanical stress sensor on or within a housing of the IMD.

FIG. 10 is a flow diagram of an example technique for determining the mechanical loads exerted on an IMD over time.

FIG. 11 is a schematic diagram illustrating a system that can be used to determine a relationship between an in-line muscle force and a normal muscle force.

FIG. 12 is a graph illustrating a plot of the normal force determined based on data from a force sensor versus the normal force determined based on an in-line force and muscle parameters.

FIG. 13 is a partial least squares plot generated using regression data from sets of data obtained from subjects of different species, where the plot demonstrates the relatively high accuracy of a transfer function (Equation 1) in identifying a relationship between an actual compressive force exerted by a muscle, as determined via a force sensor, and a compressive (or normal) force determined based on an in-line muscle force and muscle parameters.

FIG. 14 illustrates residual plots for a compressive force exerted by a muscle in a direction substantially normal to the direction in which the muscle contracts, whereby the residuals indicate a difference between the actual compressive force determined via a force sensor and a regressed function value of the compressive force determined via a transfer function (Equation 1).

FIG. 15 is a flow diagram illustrating a technique for determining a compressive force exerted by a muscle.

FIGS. 16A-16E are each a table illustrating the data that was used to generate a transfer function (Equation 1) that indicates a relationship between an in-line muscle force and a normal muscle force.

### DETAILED DESCRIPTION

Implantable medical devices (IMDs) deliver therapy to a patient to treat or otherwise manage a patient condition. Example therapies include electrical stimulation therapy, therapeutic agent delivery therapy (e.g., drug delivery, genetic material delivery or biologics delivery), or combinations thereof. Electrical stimulation therapy can include, for example, cardiac pacing therapy, delivery of defibrillation or cardioversion shocks, neurostimulation therapy, functional electrical stimulation, peripheral nerve field stimulation therapy, deep brain stimulation (DBS) therapy, and other types of therapy that include delivering electrical stimulation from an IMD to a nerve, organ, muscle, muscle group or other tissue site within a patient. Therapeutic agent delivery therapy can include, for example, delivery of one or more pharmaceutical agents, insulin, pain relieving agents, gene therapy agents or the like from an IMD to a target tissue site in a patient.

An IMD may be subject to mechanical loads before implantation in a patient, e.g., during manufacturing, shipping, and other handling of the IMD. In addition, the IMD may be subject to mechanical loads during implantation in the patient and after implantation in the patient. The mechanical loads exerted on an IMD may generate mechanical stresses within the IMD (e.g., within a housing of the IMD or a component within the housing). According to systems and techniques described in this disclosure, a mechanical stress sensor is mechanically coupled to an outer housing of the IMD or a component enclosed within the outer housing. The component may be an electrical component or a mechanical component, and may or may not aid in the delivering of therapy or monitoring of a patient parameter. For example, the component may be a therapy delivery module, a circuit board including circuitry for controlling a therapy delivery module, a power source, a memory, a sub-housing enclosed within the outer housing, or any other component that may be within an outer housing of an IMD.

The mechanical stress sensor generates a signal indicative of mechanical loads exerted on the IMD, which may be attributable to one or more various sources. Examples of possible sources of mechanical loads that may be exerted on an IMD include, but are not limited to, forces generated by tissue proximate an implanted IMD, forces generated by physiological functions of the patient in which the IMD is implanted (e.g., cardiac contractions), forces generated external to the patient and transmitted to the IMD through tissue of the patient, forces generated by movement at a joint of the patient if the IMD is implanted proximate the joint (e.g., a location at which two or more bones make contact), and the like. With respect to forces generated external to the patient, the forces may be attributable to many different types of sources. For example, the mechanical stress sensor may generate a signal indicative of a patient fall or being hit in a manner that causes a mechanical load to be exerted on the IMD (e.g., the deployment of an airbag). In this manner, the mechanical stress sensor may be useful for monitoring patient well-being. In addition, mechanical loads that are exerted on an IMD may be encountered during the manufacturing, shipping or other handling of the IMD.

Mechanical stress (e.g., internal forces) may be produced within the IMD in reaction to the external mechanical loads applied to IMD, regardless of whether the external mechanical loads are generated by forces internal or external to the patient. The mechanical stress sensor generates a signal that changes as a function of the mechanical stress. In this way, the signal generated by the mechanical stress sensor is indicative of mechanical loads exerted on the IMD. Mechanical stress may be measured in terms of the amount of exerted per unit area, which can be indicated by Pascals, Newtons per square meter, pounds per square inch, or other suitable dimensions.

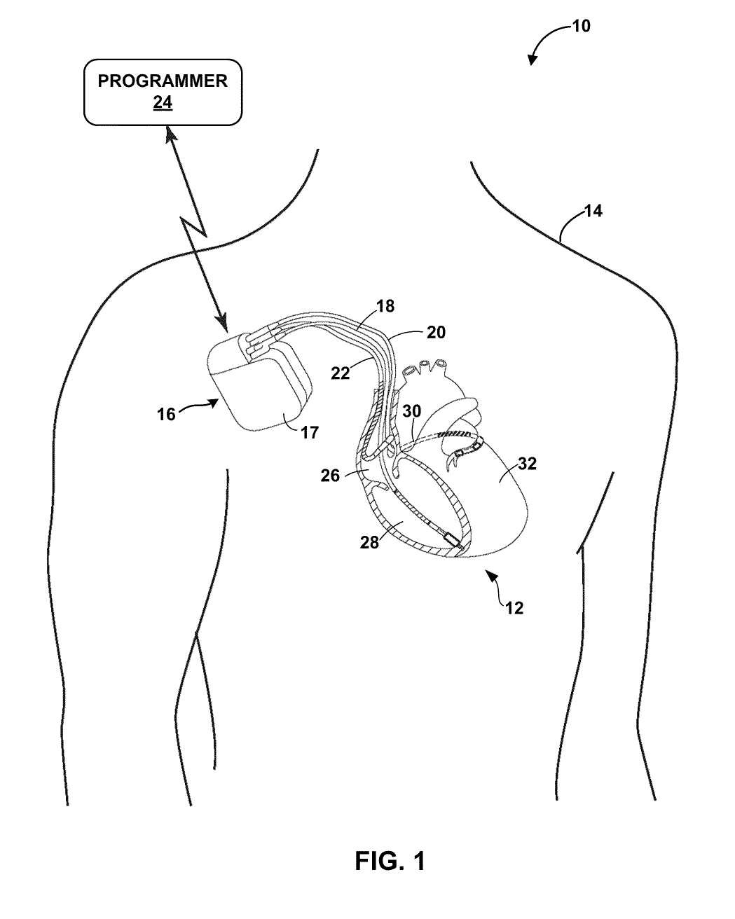

An IMD including at least one mechanical stress sensor mechanically connected to at least one of an outer housing or a component within the outer housing may be configured to deliver any suitable type of therapy to the patient. FIGS. 1-3 are conceptual diagrams of example therapy systems that include different types of IMDs that include the at least one mechanical stress sensor.

FIG. 1 is a conceptual diagram illustrating an example therapy system 10 that provides electrical stimulation therapy to heart 12 of patient 14, which may be a human patient. Therapy system 10 includes IMD 16, which is coupled to leads 18, 20, and 22, and programmer 24. IMD 16 is a medical device that provides cardiac rhythm management therapy to heart 12, and may include, for example, an implantable pacemaker, cardioverter, and/or defibrillator that provide therapy to heart 12 of patient 14 via electrodes coupled to one or more of leads 18, 20, and 22. In some examples, IMD 16 may deliver pacing pulses, but not cardioversion or defibrillation pulses, while in other examples, IMD 16 may deliver cardioversion or defibrillation shocks, but not pacing pulses. In addition, in further examples, IMD 16 may deliver pacing pulses, cardioversion shocks, and defibrillation shocks.

In some examples, IMD 16 may not deliver cardiac rhythm management therapy to heart 12, but may instead only sense electrical cardiac signals of heart 12 and/or other physiological parameters of patient 14 (e.g., blood oxygen saturation, blood pressure, temperature, heart rate, respiratory rate, muscle activity, and the like), and store the electrical cardiac signals and/or other physiological parameters of patient 14 for later analysis by a clinician. In such examples, IMD 16 may be referred to as a patient monitoring device. Examples of patient monitoring devices include, but are not limited to, the Reveal Plus Insertable Loop Recorder, which is available from Medtronic, Inc. of Minneapolis, Minn. For ease of description, IMD 16 will be referred to in this disclosure as a cardiac rhythm management therapy delivery device.

Leads 18, 20, 22 extend into the heart 12 of patient 14 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

IMD 16 may sense electrical signals attendant to the depolarization and repolarization of heart 12 via electrodes (not shown in FIG. 1) coupled to at least one of the leads 18, 20, 22. In some examples, IMD 16 may also sense electrical signals attendant to the depolarization and repolarization of heart 12 via extravascular electrodes (e.g., outside the vasculature of patient 14), such as epicardial electrodes, external surface electrodes, subcutaneous electrodes, electrodes on outer housing 17 of IMD 16 (or formed by outer housing 17), and the like. Outer housing 17 can be constructed of a biocompatible material, such as titanium or stainless steel, or a polymeric material such as silicone or polyurethane, and surgically implanted in a subcutaneous pocket near the clavicle of patient 14 or at another implant site within patient 14 (e.g., a submuscular tissue site).

In some examples, IMD 16 provides pacing pulses to heart 12 based on the electrical signals sensed within heart 12. These electrical signals sensed within heart 12 may also be referred to as cardiac signals or electrical cardiac signals. The configurations of electrodes used by IMD 16 for sensing and pacing may be unipolar or bipolar.

IMD 16 may also provide defibrillation therapy and/or cardioversion therapy via electrodes located on at least one of the leads 18, 20, 22. IMD 16 may detect arrhythmia of heart 12, such as fibrillation of ventricles 28, 32, and deliver defibrillation therapy to heart 12 in the form of electrical pulses. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

In some examples, programmer 24 may be a handheld computing device or a computer workstation. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some examples, a display of programmer 24 may include a touch screen display, and a user may interact with programmer 24 via the display.

A user, such as a physician, technician, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD.

For example, the user may use programmer 24 to retrieve information from IMD 16 regarding the rhythm of heart 12, trends therein over time, or tachyarrhythmia episodes. As another example, the user may use programmer 24 to retrieve information from IMD 16 regarding other sensed physiological parameters of patient 14, such as sensed electrical cardiac activity, intracardiac or intravascular pressure, patient activity level, patient posture, respiration rate or thoracic impedance. As another example, the user may use programmer 24 to retrieve information from IMD 16 regarding the performance or integrity of IMD 16 or other components of system 10, such as leads 18, 20, and 22, or a power source of IMD 16.

The user may use programmer 24 to program a therapy progression, select electrodes used to deliver defibrillation shocks, select waveforms for the defibrillation pulse, or select or configure a fibrillation detection algorithm for IMD 16. The user may also use programmer 24 to program aspects of other therapies provided by IMD 14, such as cardioversion or pacing therapies. In some examples, the user may activate certain features of IMD 16 by entering a single command via programmer 24, such as depression of a single key or combination of keys of a keypad or a single point-and-select action with a pointing device.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

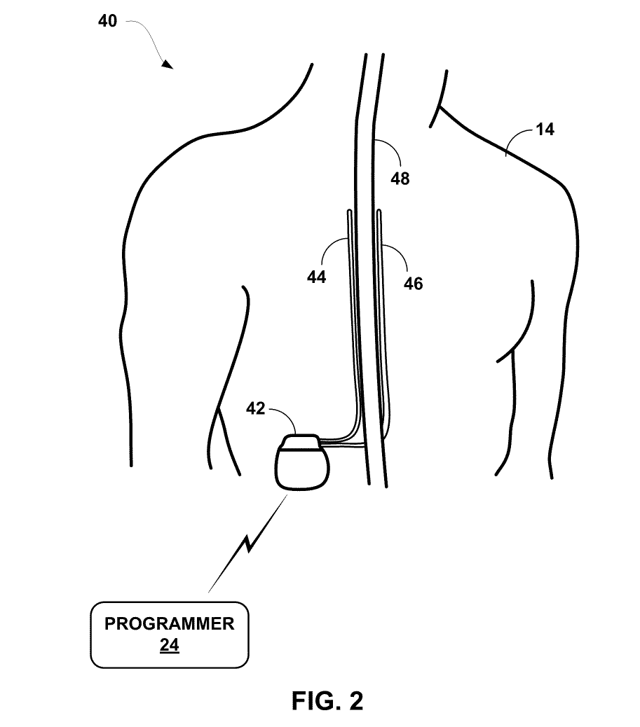

FIG. 2 is a conceptual diagram illustrating an example implantable therapy system 40 including IMD 42 and two implantable stimulation leads 44, 46 that deliver electrical stimulation therapy to a tissue site proximate spine 48 of patient 14. Therapy system 40 also includes programmer 24. In the example of FIG. 2, IMD 42 is an implantable electrical stimulator configured for spinal cord stimulation (SCS), e.g., for relief of chronic pain or other symptoms. Stimulation energy is delivered from IMD 42 to spinal cord 48 of patient 14 via one or more electrodes of implantable leads 44, 46. In some applications, such as SCS to treat chronic pain, the leads 44, 46 may be implanted such that the longitudinal axes of leads 44, 46 are substantially parallel to one another.

Each of leads 44, 46 may include electrodes (not shown in FIG. 2), and the parameters for a therapy program that controls delivery of stimulation therapy by IMD 42 may include information identifying which electrodes have been selected for delivery of stimulation according to a stimulation program, the polarities of the selected electrodes, i.e., the electrode configuration for the program, and voltage or current amplitude, pulse rate, and pulse width of stimulation delivered by the electrodes. Delivery of electrical stimulation pulses is described for purposes of illustration. However, stimulation may be delivered in other forms, such as continuous waveforms. Programs that control delivery of other therapies by IMD 42 may include other parameters, e.g., such as dosage amount, rate, or the like for drug delivery.

In the example shown in FIG. 2, leads 44, 46 carry one or more electrodes (not shown) that are placed adjacent to the target tissue of spinal cord 48 of patient 14. One or more electrodes may be disposed proximate to a distal end of a lead 44, 46 and/or at other positions at intermediate points along the leads 44, 46. Electrodes of leads 44, 46 transfer electrical stimulation generated by IMD 42 to tissue of patient 14. The electrodes may be electrode pads on a paddle lead, circular (e.g., ring) electrodes surrounding the body of leads 44, 46, conformable electrodes, cuff electrodes, segmented electrodes, or any other type of electrodes capable of forming unipolar, bipolar or multipolar electrode configurations for therapy. In general, ring electrodes arranged at different axial positions at the distal ends of leads 44, 46 will be described for purposes of illustration.

Leads 44, 46 may be implanted within patient 14 and directly or indirectly (e.g., via a lead extension) electrically connected to IMD 42. Alternatively, leads 44, 46 may be implanted and coupled to an external stimulator, e.g., through a percutaneous port. In some cases, an external stimulator may be a trial or screening stimulation that is used on a temporary basis to evaluate potential efficacy to aid in consideration of chronic implantation for a patient. In other examples, IMD 42 is a leadless stimulator with one or more arrays of electrodes arranged on a housing of the stimulator rather than leads that extend from the housing.

IMD 42 delivers electrical stimulation therapy to patient 14 via selected combinations of electrodes carried by one or both of leads 44, 46. The target tissue for the electrical stimulation therapy may be any tissue affected by electrical stimulation energy, which may be in the form of electrical stimulation pulses or continuous waveforms. In some examples, the target tissue includes nerves, smooth muscle or skeletal muscle. In the example illustrated by FIG. 2, the target tissue is tissue proximate spinal cord 48, such as within an intrathecal space or epidural space of spinal cord 48, or, in some examples, adjacent nerves that branch off of spinal cord 48. At least a portion of leads 44, 46 may be introduced into spinal cord 48 in via any suitable region, such as the thoracic, cervical or lumbar regions. Stimulation of spinal cord 48 may, for example, prevent pain signals from traveling through spinal cord 48 and to the brain of patient 14. Patient 14 may perceive the interruption of pain signals as a reduction in pain and, therefore, efficacious therapy results.

The deployment of electrodes via leads 44, 46 connected to IMD 42 is described for purposes of illustration. Arrays of electrodes may be deployed in different ways. For example, a housing associated with a leadless stimulator may carry arrays of electrodes, e.g., rows and/or columns (or other patterns). Such electrodes may be arranged as surface electrodes, ring electrodes, or protrusions. As a further alternative, electrode arrays may be formed by rows and/or columns of electrodes on one or more paddle leads. In some examples, electrode arrays may include electrode segments, which may be arranged at respective positions around a periphery of a lead, e.g., arranged in the form of one or more segmented rings around a circumference of a cylindrical lead.

In the example of FIG. 2, stimulation energy is delivered by IMD 42 to spinal cord 48 to reduce the amount of pain perceived by patient 14. However, in other examples, IMD 42 may be used with a variety of different therapies, such as peripheral nerve stimulation (PNS), peripheral nerve field stimulation (PNFS), DBS, cortical stimulation (CS), pelvic floor stimulation, gastric stimulation, and the like. The electrical stimulation delivered by IMD 42 may take the form of electrical stimulation pulses or continuous stimulation waveforms, and may be characterized by controlled voltage levels or controlled current levels, as well as pulse width and pulse rate in the case of stimulation pulses.

IMD 42 may be constructed with a biocompatible outer housing, such as titanium or stainless steel, or a polymeric material such as silicone or polyurethane, and surgically implanted at a site in patient 14 near the pelvis. In some cases, the implant site for IMD 42 may be selected to be a location in which the implanted IMD 42 is minimally noticeable to patient 14. For SCS, IMD 42 may be located in the lower abdomen, lower back, upper buttocks, or other suitable location to secure IMD 42. Leads 44, 46 may be tunneled from IMD 14 through tissue to reach the target tissue adjacent to spinal cord 48 for stimulation delivery. Alternatively, IMD 14 may be external and deliver therapy to patient 14 via percutaneously implanted leads. In other examples of therapy system 40, IMD 42 may be coupled to one lead or more than two leads (e.g., three leads).

Programmer 24 is configured to communicate with IMD 42, e.g., via wireless communication signals. A user, such as a clinician or patient 14 may interact with a user interface of external programmer 24 to program IMD 42. Programming of IMD 42 may refer generally to the generation and transfer of commands, therapy programs, or other information to control the operation of IMD 42. In the case of electrical stimulation therapy, a therapy program may be characterized by an electrode combination, electrode polarities, voltage or current amplitude, pulse width, pulse rate, and/or duration. A group may be characterized by multiple programs that are delivered simultaneously or on an interleaved or rotating basis.

Although FIG. 2 is directed to SCS therapy, system 40 may alternatively be directed to any other condition that may benefit from stimulation therapy. For example, system 40 may be used to treat movement disorders (e.g., tremor), Parkinson's disease, epilepsy, urinary or fecal incontinence, sexual dysfunction, obesity, gastroparesis, or psychiatric disorders (e.g., depression, mania, obsessive compulsive disorder, anxiety disorders, and the like). In this manner, system 40 may be configured to provide therapy taking the form of DBS, pelvic floor stimulation, gastric stimulation, or any other stimulation therapy.

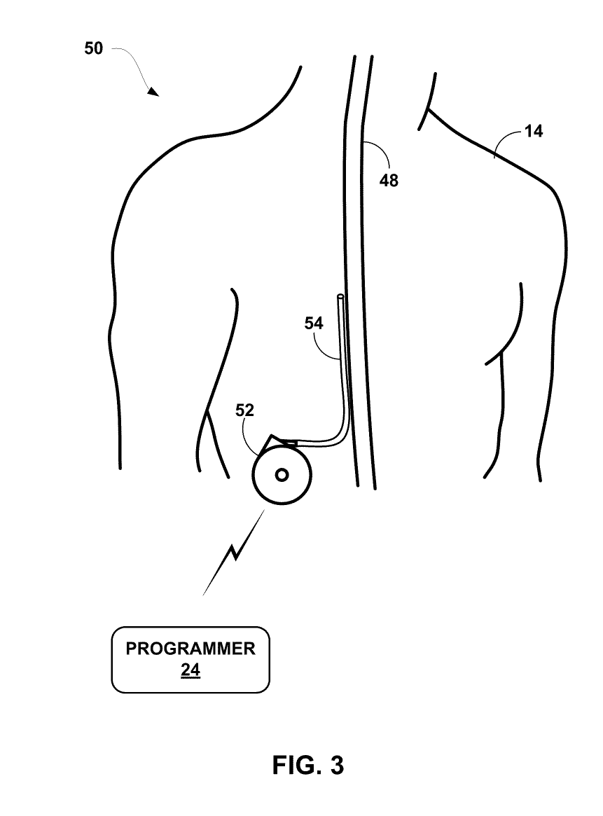

As previously indicated, in some examples, an IMD that includes a mechanical stress sensor is configured to deliver a therapeutic agent to patient 14, e.g., to manage a patient condition by minimizing or even eliminating symptoms associated with a patient condition. FIG. 3 is a conceptual diagram illustrating an implantable drug delivery system 50 including IMD 52 and drug delivery catheter 54, which is mechanically and fluidically coupled to IMD 52. As shown in the example of FIG. 3, drug delivery system 50 is substantially similar to therapy systems 10 and 40. However, drug delivery system 50 performs the similar therapy functions via delivery of therapeutic agents instead of electrical stimulation. IMD 52 functions as a drug pump in the example of FIG. 3, and IMD 52 communicates with external programmer 24 to initialize therapy or modify therapy during operation. In addition, IMD 52 may be refillable to allow chronic drug delivery.

A fluid delivery port of catheter 54 may be positioned within an intrathecal space or epidural space of spinal cord 48, or, in some examples, adjacent nerves that branch off of spinal cord 48. Although IMD 52 is shown as coupled to only one catheter 54 positioned along spinal cord 48, additional catheters may also be coupled to IMD 52. Multiple catheters may deliver drugs or other therapeutic agents to the same anatomical location or the same tissue or organ. Alternatively, each catheter may deliver therapy to different tissues within patient 14 for the purpose of treating multiple symptoms or conditions. In some examples, IMD 52 may be an external device that includes a percutaneous catheter that delivers a therapeutic agent to patient 14, e.g., in the same manner as catheter 54. Alternatively, the percutaneous catheter may be coupled to catheter 54, e.g., via a fluid coupler. In other examples, IMD 52 may include both electrical stimulation capabilities, e.g., as described with respect to IMDs 16 (FIG. 1) and 42 (FIG. 2), and drug delivery therapy.

IMD 52 may also operate using parameters that define the method of drug delivery. IMD 52 may include programs, or groups of programs, that define different delivery methods for patient 14. For example, a program that controls delivery of a drug or other therapeutic agent may include a titration rate or information controlling the timing of bolus deliveries. Patient 14 or a clinician may use external programmer 24 to adjust the programs or groups of programs to regulate the therapy delivery.

IMDs 16, 42, 52 are described in this disclosure for purposes of illustration. In other examples, other types of medical devices that deliver a therapy to patient 14 include a mechanical stress sensor to monitor the mechanical loads exerted on the IMD in accordance with the techniques described in this disclosure. In general, the IMD may be implanted in any suitable location within patient 14, such as within subcutaneous tissue (e.g., in a subcutaneous pocket) or at a submuscular location. It is believed that based on trial implantation of devices having a form factor similar to an implantable pacemaker in non-human primates (and, in particular, in a pectoral region), greater in vivo, anatomically-induced mechanical loads may be exerted on an IMD implanted in a submuscular location compared to an IMD implanted in a subcutaneous location. This may be attributable to, for example, due to the actual muscle induced mechanical loads produced during muscle exertion being force coupled to the IMD in the case of submuscular implantation and force isolated in the case of subcutaneous implantation, as well as the implant depth of the IMD. An anatomically-induced mechanical load is, for example, a force exerted on an implanted IMD by tissue from movement of patient 14. An anatomically-induced mechanical load is not attributable to a load that is generated external to patient 14.

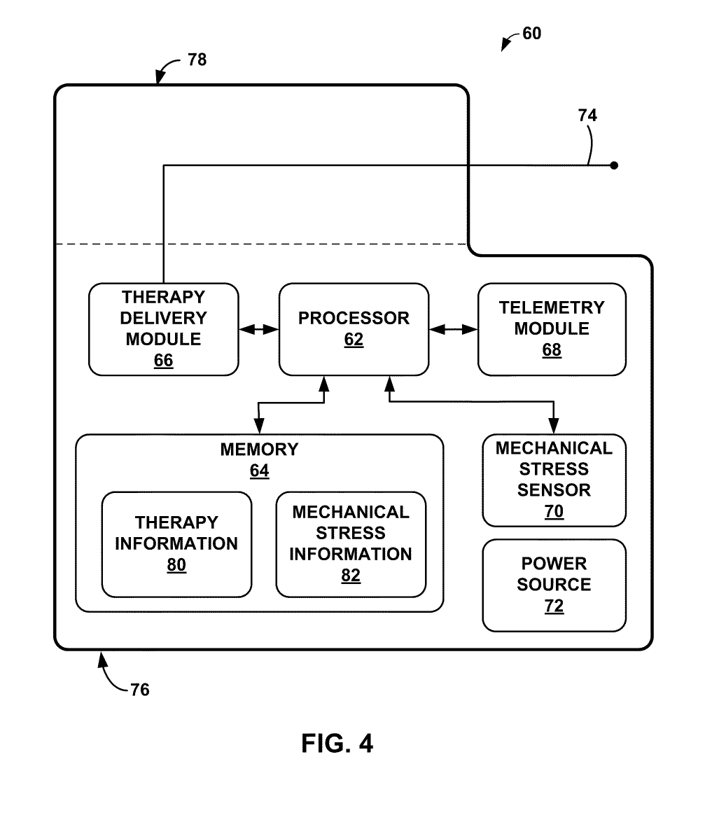

FIG. 4 is a functional block diagram of an example IMD 60, which may be, for example, IMD 16 (FIG. 1), IMD 42 (FIG. 2), IMD 52 (FIG. 3) or another IMD that delivers therapy to a patient. In the example shown in FIG. 4, IMD 60 includes a processor 62, memory 64, therapy delivery module 66, telemetry module 68, mechanical stress sensor 70, and power source 72. In some examples, IMD 60 may also include a sensing module (not shown in FIG. 4) to sense one or more physiological parameters of patient 14. Processor 62, memory 64, therapy delivery module 66, telemetry module 68, and power source 72 are substantially enclosed within outer housing 76. Outer housing 76 comprises a biocompatible material, such as titanium or biologically inert polymers. In some examples, outer housing 76 is hermetically sealed. As described in further detail below, mechanical stress sensor 70 is enclosed within outer housing 76 in some examples, and in other examples, sensor 70 is on an exterior surface of housing 76, on or within connector block 78 or on or within therapy delivery member 74 (which may also be referred to as a therapy delivery element).

Connector block 78 mechanically connects therapy delivery member 74 to IMD 60. Connector block 78 is coupled to outer housing 76 using any suitable technique. Therapy delivery member 74 may be an electrical stimulation lead, a therapeutic agent delivery catheter or any other suitable member that is configured to deliver therapy from IMD 60 to a tissue site within patient 14. Although one therapy delivery member 74 is shown in FIG. 4, in other examples, therapy delivery module 66 may be coupled to any suitable number of therapy delivery members, such as two, three, four or more, either directly or indirectly (e.g., via an extension). In addition, in some examples, IMD 60 may be a leadless or catheter-less device that delivers therapy to a patient without a therapy delivery member.

In examples in which therapy delivery member 74 comprises an electrical stimulation lead that includes one or more electrodes (e.g., near a distal end or along the length of the lead), connector block 78 includes one or more electrical contacts that electrically connect the electrodes of the lead to therapy delivery module 66. For example, a proximal end of therapy delivery member 74 may be introduced into an opening defined by connector block 78 and when properly aligned, electrical contacts at a proximal end of therapy delivery member 74 may contact and electrically connect to electrical contacts within connector block 78. Connector block 78 may also be referred to as an electrical connection assembly or a header.

In examples in which therapy delivery member 74 comprises a therapeutic agent delivery catheter, connector block 78 defines an opening that receives a proximal end of the catheter, and mechanically couples to the proximal end of the catheter. Connector block 78 can also fluidically couple the catheter to a fluid reservoir that is substantially enclosed within outer housing 76 of IMD 60. In such examples, connector block 78 may include, for example, a sealed structure through which fluid may be directly passed to the catheter.

Processor 62 controls therapy delivery module 66 to deliver therapy to patient 14 via therapy delivery member 74. In particular, processor 62 controls therapy delivery module 66 to generate and delivery therapy according to one or more therapy parameter values, which may be stored in memory 64. Components described as processors within IMD 60, external programmer 24 or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination. The functions attributed to processors described in this disclosure may be provided by a hardware device and embodied as software, firmware, hardware, or any combination thereof.

In some examples, therapy delivery module 66 includes a stimulation generator that generates electrical stimulation therapy and delivers the electrical stimulation to a target tissue site within patient 14 via one or more electrodes of the therapy delivery member 74. The stimulation generator may include stimulation generation circuitry to generate stimulation pulses or continuous waveforms, and, in some examples, a switching module to switch the stimulation across different electrode combinations, e.g., in response to control by processor 62. In particular, processor 62 may control the switching module on a selective basis to cause the stimulation generator of therapy delivery module 66 to deliver electrical stimulation to selected electrode combinations and to shift the electrical stimulation to different electrode combinations when the therapy must be delivered to a different location within patient 14. The switching module may be a switch array, switch matrix, multiplexer, or any other type of switching device suitable to selectively couple stimulation energy to selected electrodes. In other examples, the stimulation generator may include multiple current sources to drive more than one electrode combination at one time.

In other examples, therapy delivery module 66 includes a fluid pump (e.g., a drug pump), which may be a mechanism that delivers a therapeutic agent in a metered or other desired flow dosage to the therapy site within patient 14 from a reservoir within IMD 60 via the therapy delivery member 74, which may be a catheter in examples in which therapy delivery module 66 includes a fluid pump. Processor 62 controls the operation of the fluid pump with the aid of instructions that are stored in memory 64. For example, the instructions may define therapy programs that specify the bolus size of a therapeutic agent that is delivered to a target tissue site within patient 14 via therapy delivery member 74. The therapy programs may also include other therapy parameters, such as the frequency of bolus delivery, the concentration of the therapeutic agent delivered in each bolus, the type of therapeutic agent delivered if IMD 60 is configured to deliver more than one type of therapeutic agent), a lock-out time interval during which therapy delivery module 66 does not deliver a therapeutic agent to patient 14, and so forth. In some examples, IMD 60 includes both a fluid pump and an electrical stimulation generator for producing electrical stimulation in addition to delivering a therapeutic agent.

Memory 64 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Memory 64 may store instructions for execution by processor 62. For example, memory 64 can store program instructions that, when executed by processor 62, cause therapy delivery module 66 to deliver therapy (e.g., electrical stimulation therapy or drug delivery therapy) to patient 14. The program instructions may be stored as therapy information 80, which can include one or more therapy parameter values that define therapy delivery to patient 14, where the therapy parameter values may be stored as a set of therapy parameter values (e.g., a therapy program).

Memory 64 also stores mechanical stress information 82. As described in further detail below, mechanical stress information 82 may include signals generated by mechanical stress sensor 70, which indicate the mechanical forces exerted on IMD 60. Changes in mechanical stress are sensed by mechanical stress sensor 70 as loads are applied to and removed from IMD 60, whether the loads are automatically-induced or generated external to patient 14 and transferred to IMD 60 through tissue of the patient. In addition, in some examples, mechanical stress information 82 may include other information determined based on the signals generated by mechanical stress sensor 70. For example, the mechanical stress information 82 can include the average mechanical stress (e.g., a value indicative of the average amount of force exerted per unit area, which can be indicated by Pascals, Newtons per square meter, pounds per square inch, or the like) sensed by mechanical stress sensor 70 for a predetermined duration of time (e.g., hourly, daily, weekly or the like), the minimum or maximum stress sensed by mechanical stress sensor 70 during a predetermined duration of time, a range of mechanical stresses sensed by sensor, the date and time at which a mechanical stress sensed by sensor 70 was greater than or equal to a threshold value, a count of the number of times a sensed mechanical stress exceeded a predetermined threshold value and the duration of the amount of time the mechanical stress remained above the threshold, and the like. The predetermined threshold value may indicate a transient or cumulative stress value. Raw signals from sensor 70 or processed values may be stored in memory 82. Processing of the raw signals generated by mechanical stress sensor 70 to produce the processed values may be done in an external programmer 24, server, or another external device.

In some examples, patient 14 may provide input (e.g., via programmer 24) indicating an activity or posture undertaken by patient 14 when a particular mechanical load was sensed by sensor 70. IMD 60 or programmer 24 may prompt patient 14 for the activity or posture information or patient 14 may periodically provide the information on the patient's own volition. In such examples, mechanical stress information 82 can also include the activity or posture indicated by patient 14 and associated with the mechanical stress sensed by sensor 70. The activity or posture information may be generated by a separate sensor, such as an accelerometer (e.g., a two-axis or a three-axis accelerometer) and the information from the separate sensor can be correlated with the force/stress information.

Therapy information 80 may include any data created by or stored in IMD 60, as well as therapy parameter values for controlling therapy generation and delivery by therapy module 66. Therapy information 80 and/or mechanical stress information 82 may be recorded for long-term storage and retrieval by a user. In the example shown in FIG. 4, memory 64 stores therapy information 80 and mechanical stress information 82 in separate memories of memory 64 or separate areas within memory 64.

In some examples, sensor 70 is mechanically coupled to at least one of outer housing 76, connector block 78, a component within outer housing 76 or a component within connector block 78. As previously indicated, a component may be an electrical component or a mechanical component. For example, the component may be processor 62, memory 64, therapy delivery module 66, telemetry module 68, power source 72, a sub-housing of each of the aforementioned components, or any other component that may be within outer housing 76 of IMD or connector block 78.

In some examples, mechanical stress sensor 70 is directly or indirectly mechanically connected to a surface of outer housing 76 (e.g., an interior or an exterior surface of outer housing 76, whereby the exterior surface can be configured to contact tissue of patient 14 when IMD 60 is implanted within patient 14) or an interior or exterior surface of connector block 78. In other examples, mechanical stress sensor 70 is directly or indirectly mechanically connected to a component that is substantially enclosed within outer housing 76 or connector block 78. For example, in some examples, mechanical stress sensor 70 is mechanically mounted on a circuit board that is enclosed within housing 76 or to power source 72 (e.g., a housing of power source 72 that is separate from outer housing 76 of IMD 60). A mechanical coupling between sensor 70 and housing 76, connector block 78 or a component within housing 76 or connector block 78 may be accomplished using any suitable technique, such as a pressure sensitive adhesive or another type of adhesive, solder reflow, or other suitable mechanical attachment mechanisms.

In other examples, rather than being a separate component that is attached to IMD 60, mechanical stress sensor 70 can be integrally formed with a component of IMD 60, housing 76 or connector block 78. For example, a mechanical stress sensing material (e.g., a resistive material) could be integrated into a component of IMD 60 or a therapy delivery member 74 via extrusion or coating process or a mechanical stress sensor may be laminated directly into a composite structure (whether the composite structure is a part of housing 76, connector block 78, therapy delivery member 74 or a component within housing 76, connector block 78 or therapy delivery member 74).

As another example, sensor 70 can be etched into a circuit board that is enclosed within housing 76. As an example, sensor 70 may comprise a resistive component (e.g., a resistive trace or ink) that is integrated into a printed circuit board. As another example, sensor 70 can be formed by a resistive material that is fabricated within a wall of housing 76 or connector block 78 (e.g., etched into an exterior or interior surface of housing 76 or connector block 78). A sensor 70 including resistive ink or a resistive trace may change resistance as a mechanical load is applied to IMD 60, thereby indicating the mechanical stress exerted on IMD 60.

In addition to or instead of sensor 70 on or within housing 76 or connector block 78, in some examples, mechanical stress sensor 70 can be mechanically coupled to or integrated within therapy delivery member 74 (e.g., proximate a distal end of therapy delivery member 74). In this configuration, sensor 70 may generate a signal indicative of a mechanical force exerted on therapy delivery member 74, and the signal may potentially provide a therapy indication or indication that therapy delivery member 74 has been exposed to an elevated stress level or cumulative fatigue stress levels over time. The stress conditions of therapy delivery member 74 may be attributable to various factors, such as how therapy delivery member 74 is implanted within patient 14 (e.g., due to the hemostat usage, manual bending of therapy delivery member 74 during implantation in patient 14, tensile or compressive forces resulting from the final implant configuration if therapy delivery member 74 in tissue, and the like). In this way, mechanical stress sensor 70 mechanically coupled to therapy delivery member 74 may be useful for monitoring the mechanical status, and, in some cases, the operational status, of therapy deliver element 74. In general, the signal generated by mechanical stress sensor 70 during implantation of therapy system 10 in patient 14 may be useful for monitoring the implant handling techniques of the element to which sensor 70 is attached.

Mechanical stress sensor 70 generates a signal indicative of a mechanical force exerted on IMD 60 by internally or externally induced forces. In the case of an internally induced force, the mechanical force may be an anatomically-induced force that is exerted on IMD 60 by tissue proximate to IMD 60 when IMD 60 is implanted within patient 14. As a result of biomechanical properties of the tissue, such as the elastic and visco-elastic properties of tissue, different forces may be exerted on IMD 60 while IMD 60 is implanted within patient 14. As patient 14 occupies different postures or undergoes movements, e.g., as part of the daily activities, the tissue proximate to IMD 60 may exert a mechanical load on IMD 60 as a result of the deformation of tissue proximate to the implanted IMD 60. The anatomically-induced forces exerted on an implanted IMD 60 may differ depending on the activity or posture undertaken by patient 14, which may change the force that tissue adjacent IMD 60 applies to IMD 60.

Sensor 70 can also generate a signal indicative of an externally induced force that is exerted on IMD 60. The externally induced force can include, for example, a force generated external to patient 14 and transmitted through tissue of patient 14 to IMD 60.

Mechanical stress sensor 70 generates a signal that is indicative of different types of information than a signal generated by an accelerometer, which may also be included in or on IMD 60. An accelerometer generates a signal indicative of acceleration of the accelerometer, which may result from movement of patient 14. In addition, the signal generated by an accelerometer may indicate an orientation of the accelerometer in space (e.g., a three-axis accelerometer may indicate an orientation of the accelerometer within three-dimensional space), which may indicate a patient posture. While a signal generated by an accelerometer on or within IMD 60 can indicate an application of a dynamic load (e.g., a shock) to IMD 60, e.g., resulting from the movement of the IMD 60, the acceleration signal may not be indicative of other types of mechanical forces, such as static or quasi-static loads. For example, an accelerometer may not indicate when a sustained shear force or static axial force is applied to IMD 60. Moreover, the accelerometer may not provide information indicative of a mass that is applied to IMD 60. In contrast to the accelerometer, sensor 70 is configured to generate a signal that changes as a function of a static mechanical force that is applied to IMD 60. The static mechanical force that is applied to IMD 60 changes the stress sensed by sensor 70, and, therefore, varies the signal generated by sensor 70. For at least these reasons, sensor 70 may provide a more robust indication of a mechanical load acting on IMD 60 than an accelerometer.

In some examples, mechanical stress sensor 70 includes a single sensor. In other examples, mechanical stress sensor 70 comprises a plurality of sensors that are physically separate from each other and distributed about IMD 60. For example, IMD 60 may include up to 10 or more mechanical stress sensors. In such examples, separate load sensors may be mechanically connected to housing 76, connector block 78, and/or one or more components substantially enclosed within outer housing 76 or connector block 78. Processor 62 may include separate channels for receiving signals from each of the plurality of mechanical stress sensors. A plurality of sensors distributed at different locations within or on housing 78 or connector block 78 may be useful for monitoring the stresses exerted at different components of IMD 60, as well as at different portions of IMD 60. In addition, a plurality of mechanical stress sensors 70 may indicate a distribution of the mechanical loads exerted on IMD 60.

Mechanical stress sensor 70 can be configured to convert a mechanical load exerted on the sensor to an electrical signal that is received by processor 62. In this way, the electrical signal generated by mechanical stress sensor 70 may be indicative of a mechanical load exerted on IMD 60, whether the load is an axial load, a shear stress or any combination thereof. As described in further detail below, in some examples, processor 62 receives a signal generated by mechanical stress sensor 70 and determines a mechanical load exerted on IMD 60 or a mechanical stress induced within IMD 60 at a particular time or over a period of time based on the signal. Processor 62 may determine the mechanical load by determining a change in force or other applied load to IMD 60 based on the signal generated by sensor 70. A gross load value may be determined based on a comparison of the signal generated by sensor 70 to a baseline signal, which may be determined when a load is not being applied to IMD 60. While a signal generated by sensor 70 is indicative of a mechanical stress sensed by sensor 70, the mechanical stress may be correlated to a mechanical load value. The baseline signal indicative of a minimal load application to IMD 60 may be determined by the manufacturer of the sensor 70 or by a clinician at the time sensor 70 is implanted in patient 14. In some cases, an amplifier amplifies the signal generated by mechanical stress sensor 70 before processor 62 receives the signal.

In some examples, mechanical stress sensor 70 exhibits a change in electrical resistance as an external mechanical load is exerted on IMD 60. For example, mechanical stress sensor 70 may include a strain gauge that deforms in response to an applied mechanical load and changes resistance as a result of the deformation. Processor 62 may be electrically coupled to mechanical stress sensor 70 and determine the electrical resistance exhibited by mechanical stress sensor 70 or a change in electrical resistance in order to determine the mechanical load that is exerted on IMD 60 (e.g., a gross mechanical stress value or a change in mechanical stress). In some cases, the change in resistance is measured using a Wheatstone bridge, although other resistance measuring members may be used in other examples. The change in resistance or the gross resistance value may be directly related to the strain exerted on IMD 60.

Mechanical stress sensor 70 comprises any one or more different types of sensors, such as one or more strain gauges or pressure sensors. Example of suitable mechanical stress sensors include, but are not limited to, a flexible printed circuit comprising pressure sensitive ink, a piezoresistor, a piezoelectric crystal, a capacitive sensor, a load cell, a pressure transducer (e.g., a capacitive, resistive or optical pressure transducer), a force sensor, a displacement sensor or other types of analog resistance or voltage based sensors.

A flexible printed circuit may be useful for incorporating on or within a housing of IMD 60 because of its relatively small size and flexibility, which enables the circuit to adapt to different surface profiles. A mechanical stress sensor 70 including a pressure sensitive ink may exhibit decreased resistance with an increased compressive load. In some examples, sensor 70 includes a plurality of flexible printed circuits that are each configured to generate a signal indicative of an applied load by using a voltage divider circuit that produces an analog voltage that is a function of the force applied to the sensor. In some cases, the analog voltage generated by the voltage divider circuit of sensor 70 may be converted to a digital signal with an analog-to-digital converter. An example of a suitable mechanical load sensor that may be used with IMD 60 includes a flexible circuit comprising a pressure sensitive ink that is made available by Tekscan Incorporated of South Boston, Mass. under the FlexiForce trademark.

Sensor 70 may be selected to be able to withstand the environmental conditions resulting from implantation within a living patient 14, which may be a human patient. For example, sensor 70 may be selected to be able to function in an operating environment having a temperature near the body temperature of a human patient. In some cases, a mechanical force sensor may exhibit an upward voltage bias trend due to the combination of moisture and static continuous loading in vivo. In such cases, the implanted mechanical force sensor may be periodically calibrated to account for the expected upward voltage bias trend.

Additional considerations for selecting sensor 70 include an ability to withstand expected manufacturing, shipping, and handling extremes to which IMD 60 may be subject. In examples in which sensor 70 is mechanically coupled to an exterior surface of housing 76 or connector block 78, sensor 70 is selected to withstand proper sterilization before implantation within patient 14. Given the cyclic loading on IMD 60 when implanted within a living and moving patient 14, sensor 70 is selected to withstand the cyclic loading and remain substantially accurate after exposure to cyclic loading.

After implanting IMD 60 in patient 14, tissue may grow around outer housing 76, connector block 78, and therapy delivery member 74. This tissue ingrowth may be referred to as fibrous encapsulation. Fibrous encapsulation around IMD 60 when IMD 60 is implanted within patient 14 may not significantly affect the measurement of mechanical loads that are exerted on IMD 60 with stress sensor 70. It has been found, based on trials of test devices on a plurality of nonhuman, living subjects, fibrous encapsulation around the test device including a mechanical load sensor was not a discernable factor affecting the measurement of the mechanical load via the implanted mechanical stress sensor. However, the fibrous encapsulation may dampen or redistribute a mechanical force that is applied to housing 76 or connector block 78 of IMD 60.

The results of the trialing of the test devices on the living, nonhuman subjects also indicate that a known mechanical load exerted on a device that includes a plurality of mechanical stress sensors (and, in particular, flexible printed ink circuits) within an outer housing and implanted within the nonhuman subject substantially correlates to the mechanical load determined based on a signal generated by a mechanical stress sensor mechanically connected to the device.

The test device that was trialed on the living, nonhuman subjects did not include therapy delivery capabilities. However, the geometry of the device was similar to an IMD that is configured to deliver therapy to patient 14. In particular, the test device had a substantially similar geometric configuration as the EnRhythm Pacemaker made available by Medtronic, Inc. of Minneapolis, Minn. and included a plurality of FlexiForce mechanical stress sensors (made available by Tekscan Incorporated of South Boston, Mass.). The test device had a thickness of approximately 10 millimeters and each of the plurality of sensors of the test device had a maximum force rating of about 10 pounds.

Due to the dampening forces of tissue on an external load that is applied to an implanted IMD 60, the load sensed by mechanical stress sensor 70 implanted within patient 14 may not be equal to the load that is externally applied to IMD 60. The trialing of the test device on the living, nonhuman subjects indicate that tissue surrounding the implanted test device dampens an external load that is exerted via manual compressions near the implant site of the test device about 30% to about 50%. After further study, it is believed that the tissue itself does not dampen but dissipates the force laterally to the implanted test device, which being relatively unconstrained in soft tissue, moves out of the line of compression.

Processor 62 or a processor of another device (e.g., programmer 24) may monitor the force exerted on IMD 60 based on a signal generated by mechanical stress sensor 70 to monitor, and, in some cases, evaluate, the environmental conditions to which IMD 60 is exposed. In some examples, processor 62 (or the processor of another device) determines the mechanical stress to which IMD 60 is exposed based on the signal generated by mechanical stress sensor 70 and automatically stores the mechanical stress information 82 in memory 64 of IMD 60 or a memory of another device. The stored information may be, for example, the signal generated by mechanical stress sensor 70 or a value or other indication derived from the signal.

Determining the mechanical loads to which housing 76, connector block 78, and/or a component within housing 76 or connector block 78 are subjected to while implanted within patient 14, prior to implantation within patient 14, or after explantation from patient 14 may be useful for various purposes. For example, an observation of the loads to which IMD 60 is exposed can aid in the design of a more robust IMD that is configured to withstand the actually observed loads. The design criteria for an IMD that may be modified based on the mechanical stress information can include, for example, the strength of the mechanical and electrical interconnects (e.g., solder joints) between electrical components and a circuit board enclosed within housing 76 of IMD 60 or the thickness of the material with which housing 76 is formed.

The mechanical loads to which an IMD is exposed throughout its life cycle, whether implanted in patient 14 or prior to implant in patient 14, may be determined based on the mechanical stress information 82 generated by a single IMD 60 implanted in a single patient or based on a plurality of patients in which IMDs having substantially similar configurations are implanted. The mechanical loads exerted on an IMD when implanted within a living subject may be geometrically dependent because the interface between tissue and the IMD may affect the loads exerted on the IMD. Thus, if mechanical stress information is compiled for a plurality of IMDs implanted in respective patients, it may be useful to compile and compare mechanical stress information for IMDs having substantially similar, if not identical, form factors (e.g., foot print and dimensions) when determining one or more design criteria for an IMD (e.g., a future generations of an IMD).

IMD 60 including sensor 70 can be used to provide mechanical stress information that provides an understanding of the in vivo loading conditions to which IMD 60 may be exposed. The loading conditions include the magnitude and frequency of mechanical loads exerted on IMD 60 when IMD 60 is implanted in a living and moving patient. The in vivo mechanical stresses to which IMD 60 is exposed can be automatically evaluated by processor 62 while IMD 60 is implanted within patient 14 (e.g., an in vivo evaluation of stresses) or a clinician may periodically interrogate the implanted IMD 60 to retrieve mechanical stress information 82 and assess the mechanical stresses to which IMD 60 is exposed. In addition, in some examples, the clinician does not receive the mechanical stress information 82 until after IMD 60 is explanted from patient 14.

During the design of IMD 60, a clinician, engineer or other practitioner may use modeling software executing on a computing device to evaluate the impact of in vivo mechanical loads on the components of an IMD when the IMD is implanted in a patient. Determining real world in vivo conditions of an implanted IMD 60 may be useful for selecting or verifying the parameters that are used to model the in vivo conditions on the computing device. Mechanical stress sensor 70 may be useful for determining the parameters for the modeling performed by the computing device. For example, the maximum expected in vivo stress or an expected range of in vivo mechanical stresses may be determined based on the stress information generated by mechanical stress sensor 70.

In some examples, the in vivo stress condition parameters for the modeling software are determined (e.g., for updating or confirming the parameters used by the modeling software) based on information generated by a plurality of mechanical stress sensors that are mechanically connected to a respective one of a plurality of IMDs. A clinician may confirm that the computer modeling software accounts for a real world maximum mechanical load exerted on an IMD that was implanted in a patient. The IMDs that are implanted in respective patients to acquire in vivo mechanical load information may or may not have therapy delivery capabilities. For example, in some cases, a mechanical stress indicator system that is not configured to deliver therapy to a patient may be used to acquire in vivo mechanical load information. In other examples, a mechanical stress indicator system that is a part of an IMD that is configured to deliver therapy to a patient may be used to acquire in vivo mechanical load information. In general, the mechanical stress indicator system may have an outer housing that defines a form factor of an IMD for which the in vivo mechanical load information is used as an input to the design process.

Monitoring the in vivo mechanical stresses that are exerted on IMD 60 may also be useful for real-time monitoring of the conditions to which IMD 60 is exposed. For example, while IMD 60 is implanted within patient 14 and providing patient 14 with therapy or sensing a patient parameter (e.g., cardiac activity), instead of or in addition to storing the mechanical stress information 82 in memory 64 (or a memory of another device), processor 62 or the processor of another device may determine the mechanical stress to which an implanted IMD 60 is exposed based on the signal generated by mechanical stress sensor 70. Processor 62 (or another processor) may then determine whether the determined stress is indicative of a system integrity issue.

For example, if, while IMD 60 is implanted within patient 14 and providing therapy to patient 14, mechanical stress sensor 70 senses a mechanical stress that is greater than or equal to a predetermined threshold value, IMD 60 may transmit an indication to patient 14 directly or via an external device (e.g., programmer 24) to notify patient 14 or a patient caretaker that clinician attention may be desirable. The threshold value may be, for example, a transient or cumulative stress value that is indicative of the mechanical loads that indicate a compromised function of IMD 60 or therapy delivery member 74.

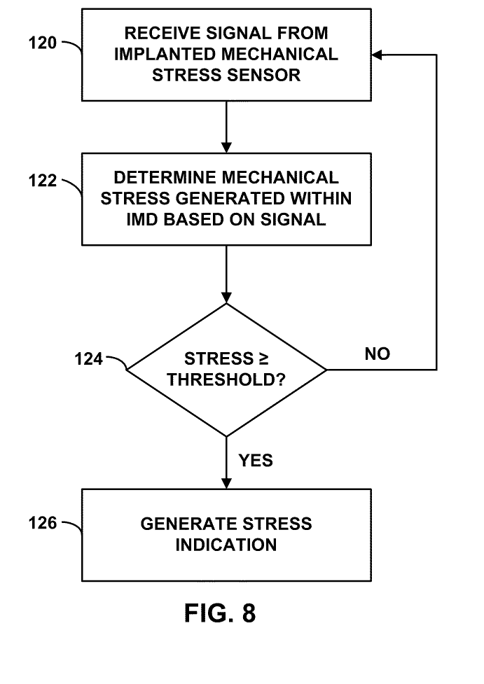

In some examples, the threshold value is a mechanical stress value that is indicative of a mechanical stress at which a mechanical connection (e.g., a solder joint) between an electrical component and a printed circuit board is compromised (e.g., at least partially fractured) or a threshold stress level at which housing 76 or connector block 78 fractures. As another example, the threshold value may be indicative of a mechanical load that has been applied to IMD 60 and is greater than or equal to an acceptable mechanical load for maintaining the structural or operational integrity of at least one component of IMD 60. An example technique for monitoring the mechanical stress indicated by mechanical stress sensor 70 and generating an indication when the mechanical stress exhibits a predetermined characteristic (e.g., a value at or above a predetermined threshold) is described with respect to FIG. 8.

As another example, if mechanical stress sensor 70 is activated prior to implantation of IMD 60 within patient 14, the signal generated by sensor 70 and stored by memory 64 or a memory of another device (e.g., programmer 24) may be used to determine the mechanical loads exerted on IMD 60 prior to implantation within patient 14. For example, a clinician may upload the mechanical stress information 82 and determine the loads that were exerted on IMD 60 during the manufacturing, transport or other handling of IMD 60. In addition, the signal generated by sensor 70 during implantation of IMD 60 therapy delivery member 74 (in examples in which a mechanical stress sensor 70 is mechanically coupled to therapy delivery member 74) in patient 14 may be useful for monitoring the implant techniques that may cause forces to be exerted on IMD 60 and/or therapy delivery member 74. For example, the signal generated by sensor 70 may be evaluated to determine whether therapy delivery member 74 was implanted in patient 14 in a manner that causes an elevated stress condition on therapy delivery member 74. The loads that are exerted on IMD 60 and therapy delivery member 74 during implantation in patient 14 may be attributable to various sources, such as the use of hemostats, manual bending of therapy delivery member 74 by the clinician, as well as tensile or compressive forces resulting from the handling of IMD 60 and/or therapy delivery member 74.

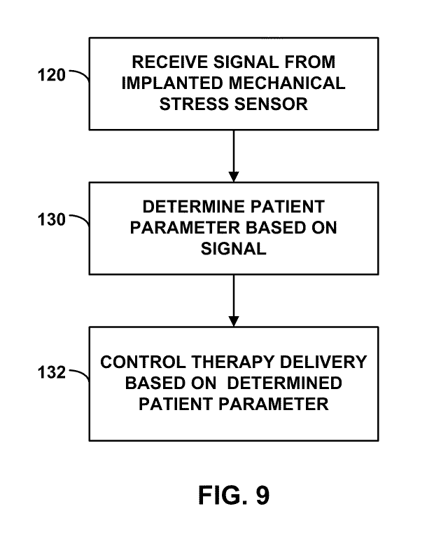

In other examples, the information generated based on the signal generated by mechanical stress sensor 70 may also be used for determining one or more parameters of patient 14. Mechanical forces may be exerted on IMD 60 by proximate tissue as a result of a patient parameter, such as a physiological parameter or patient motion. In some examples, processor 62 or a processor of another device (e.g., programmer 24) can monitor the force exerted on IMD 60 based on a signal generated by mechanical stress sensor 70 to automatically determine one or more parameters of patient 14. As described in further detail with respect to FIG. 9, processor 62 (or a processor of another device) can control therapy delivery to patient 14 based on the determined patient parameters.

Examples of physiological parameters that may be determined based on a signal generated by mechanical stress sensor 70 include, but are not limited to, muscle activity, heart rate, tissue perfusion, the presence or absence of cardiac contractions, neurological activity, and other physiological parameters that may change the in vivo mechanical load that is exerted on IMD 60. Depending on the implant site of IMD 60 within patient 14, mechanical stress sensor 70 may generate a signal that changes as a function of contraction of heart 12 (FIG. 1). The mechanical motion of heart 12 or a change in blood flow through tissue resulting from cardiac contractions may result in a changing mechanical force on IMD 60 from adjacent tissue. Processor 62 of IMD 60 (or a processor of another device) may detect the change in mechanical force exerted on housing 76 or connector block 78 resulting from the cardiac activity based on the signal generated by sensor 70. In this way, the signal from sensor 70 may be used to determine whether or when heart 12 of patient 14 is contracting. In examples in which a mechanical stress sensor 70 is mechanically coupled to therapy delivery member 74 (FIG. 4) and therapy delivery member 74 is implanted proximate to heart 12 (FIG. 1) of patient 14, the signal generated by sensor 70 may be useful for distinguishing between proper and improper cardiac function, e.g., as indicated by the timing or even the presence of cardiac contractions.

Detection of mechanical heart contractions may be useful for monitoring various physiological parameters, such as a heart rate. In addition, detecting mechanical contraction of heart 12 may be useful for confirming that heart 12 is contracting independently of electrical cardiac signals, which, in turn, may be useful for detecting electromechanical disassociation of heart 12. In addition, the magnitude of the mechanical load changes detected by sensor 70 may be indicative of intracardial timing (e.g., timing of various cardiac functions), chamber synchrony, or intracardiac pressure.

Mechanical loading information generated based on the signal generated by mechanical stress sensor 70 may also be used to determine the force exerted at a particular joint of patient 14 (e.g., a knee joint or a specific thoracic joint). If, for example, IMD 60 is implanted proximate a joint (e.g., a location where two or more bones of patient 14 contact each other directly or indirectly), a force may be exerted on IMD 60 (or therapy delivery member 74 if sensor 70 is mechanically coupled to member 74) as the bones defining the joint articulate. Example joints for which sensor 70 may be used to determine a joint force measurement include, but are not limited to, an elbow joint, a wrist joint, a knee joint, a hip joint, or vertebral joints.

Patient parameters such as patient posture or activity may also be determined based on the signal generated by mechanical stress sensor 70. For example, tissue adjacent IMD 60 may apply different loads to IMD 60 based on the patient posture or activity level, and, thus, the stress indicated by sensor 70 may be associated with a particular patient posture state or activity level. The associations between the patient posture state or activity level and one or more signal characteristics of a signal generated by sensor 70 may be predetermined, e.g., during a programming session when patient 14 is known to be in a particular patient posture state or activity level.

Mechanical stress information 82 generated based on a signal generated by mechanical stress sensor 70 may be useful for monitoring physiological patient parameters as well as a patient condition that may affect the patient's well-being. For example, for relatively immobile patients, it may be useful to monitor the pressure being applied at one or more parts of the patient's body in order to monitor for conditions in which a pressure ulcer (e.g., bedsore) may form. Pressure ulcers may be caused by unrelieved pressure, shearing forces or the like exerted on the patient's body for a certain period of time. Processor 62 of IMD 60 or a processor of another device may determine the mechanical loading conditions at a target tissue site based on the signal generated by sensor 70 and automatically monitor for conditions in which pressure ulcers are likely to form. The target tissue site can be, for example, the implant site of IMD 60 or a site of therapy delivery member 74 of sensor 70 is on the member 74.

In some examples, memory 64 stores a threshold mechanical load value (e.g., a pressure or a shear force value) under which a pressure ulcer is likely to form over a threshold duration of time. The threshold duration of time can indicate a minimum duration of time that the mechanical load must be applied to the tissue before a condition in which a pressure ulcer is likely to form is considered to be present. Processor 62 may compare a received signal from sensor 70 to the threshold values to determine when the pressure ulcer conditions are detected.

Processor 62 may generate a notification to patient 14 or a patient caretaker upon detecting a mechanical load greater than or equal to the threshold mechanical load and exerted on IMD 60 substantially continuously for a duration of time greater than or equal to the threshold duration of time. In other examples, processor 62 may generate a notification to patient 14 or a patient caretaker upon detecting a load greater than or equal to the threshold mechanical load and exerted on IMD 60 for at least a total duration of time that is greater than or equal to the threshold duration of time, where the mechanical load need not be continuously applied to IMD 60. Instead, pressure ulcer conditions may be detected when the mechanical load greater than or equal to the threshold mechanical load is applied to IMD 60 for a total period of time is greater than or equal to the threshold duration of time during a sample period of time.

Processor 62 may generate the notification by, for example, causing IMD 60 to vibrate (e.g., in a particular pattern as indicated by the intensity or timing of the vibration) or by transmitting a signal to programmer 24 or another external device. The programmer or other external device may generate an auditory, visual or somatosensory alert to notify patient 14 or the patient caretaker that pressure ulcer conditions have been detected. In response to receiving the notification, patient 14 or the patient caretaker may initiate a change in the patient's position to help relieve the pressure or other forces being applied to patient 14 at the portion of the patient's body near IMD 60.

Processor 62 controls telemetry module 68 to exchange information with another implanted or external device, such as programmer 24 (FIG. 1), by wireless telemetry. Telemetry module 68 may accomplish wireless communication with another device by RF communication techniques or via proximal inductive interaction of IMD 60 with external programmer 24. Accordingly, telemetry module 68 may send information (e.g., sensed physiological parameter information or mechanical stress information 78) to external programmer 24 on a continuous basis, at periodic intervals, or upon request from programmer 24.

Processor 62, therapy delivery module 66, mechanical stress sensor 70, and other components of IMD 60 may be coupled to power source 72. Power source 72 may take the form of a small, rechargeable or non-rechargeable battery, or an inductive power interface that transcutaneously receives inductively coupled energy. In the case of a rechargeable battery, power source 47 similarly may include an inductive power interface for transcutaneous transfer of recharge power.

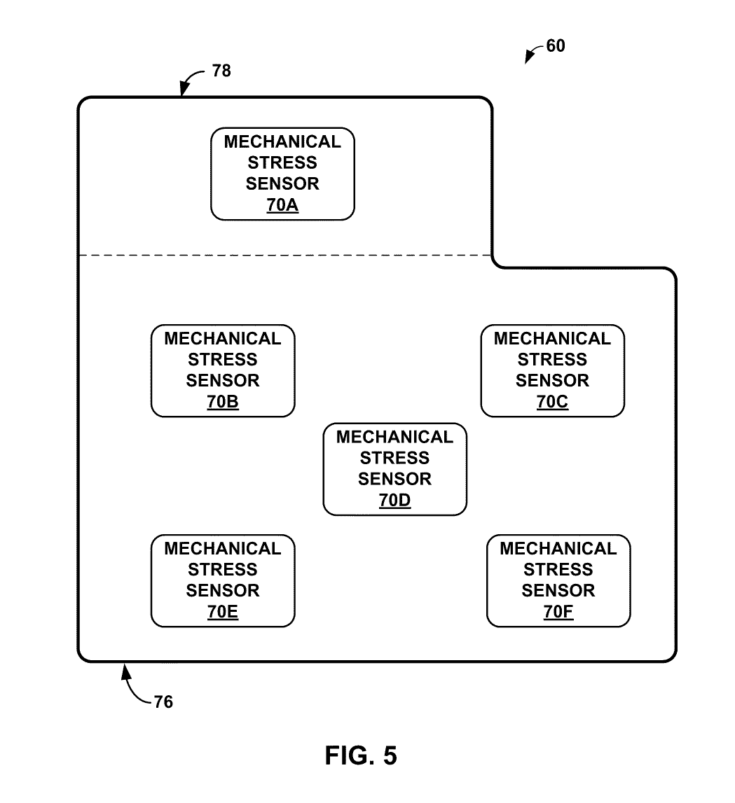

IMD 60 may include one mechanical stress sensor 70 or a plurality of mechanical stress sensors, which can be distributed about housing 76 or connector block 78 of IMD 60. FIG. 5 is a functional block diagram of an example IMD 60 that includes a plurality of mechanical stress sensors 70A-70F that are physically separate from each other. Although seven sensors 70A-70F are shown in FIG. 5, in other examples, an IMD may include any suitable number of mechanical stress sensors. Mechanical stress sensors 70A-70F may each be similar to mechanical stress sensor 70, which is described with respect to FIG. 4. Mechanical stress sensors 70A-70F may each be mechanically connected to an inner (e.g., facing the interior space defined by housing 76) or outer surface of housing 76, an inner or outer surface of connector block 78, or one or more components substantially enclosed within outer housing 76 or connector block 78.

As previously described, a plurality of physically separate mechanical stress sensors 70A-70F that are distributed about IMD 60 may be useful for monitoring the stresses exerted at different components of IMD 60, as well as at different portions of IMD 60. In addition, a plurality of mechanical stress sensors 70 may indicate a distribution of the mechanical loads exerted on IMD 60. Processor 62 may receive signals generated by each of mechanical stress sensors 70A-70F and determine a mechanical load exerted on IMD 60 or otherwise determine the mechanical stress conditions of IMD 60 based on the signal from one or more of the sensors 70A-70F. For example, processor 62 may determine the mechanical load at a particular position of IMD 60 based on a signal from an individual mechanical stress sensor 70A-70F. As another example, processor 62 may determine the general loading condition based on signals from more than one mechanical stress sensor 70A-70F. For example, processor 62 may average the signals from more than one mechanical stress sensor 70A-70F to determine the average distributed mechanical load on IMD 60. In some examples, the average may be a weighted average, whereby some mechanical stress sensors 70A-70F are attributed more weight than others. Other techniques for determining a mechanical stress on IMD 60 may be determined based on mechanical stress sensor 70A-70F.

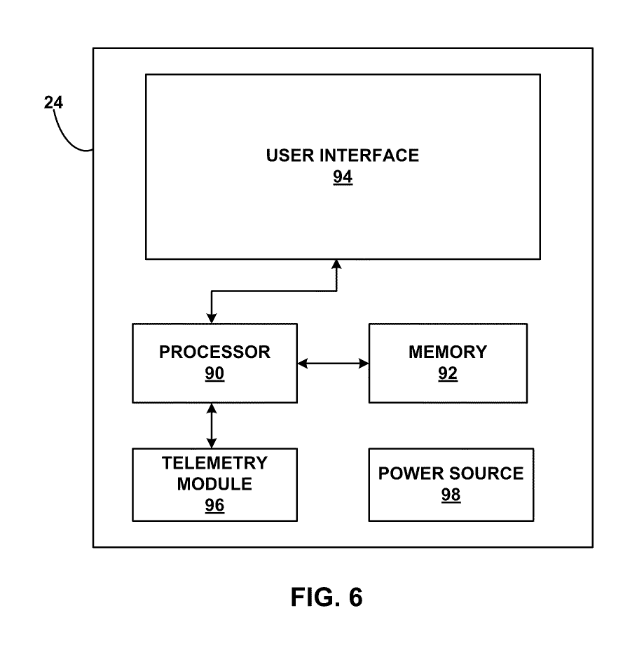

FIG. 6 is a functional block diagram of an example of programmer 24. As shown in FIG. 3, external programmer 24 includes processor 90, memory 92, user interface 94, telemetry module 96, and power source 98. A clinician or another user may interact with programmer 24 to generate and/or select therapy programs for delivery of therapy by IMD 60, which, as described above, can be an IMD that delivers electrical stimulation therapy, drug delivery therapy or other types of therapy.

Programmer 24 may be a handheld computing device, a workstation or another dedicated or multifunction computing device. For example, programmer 24 may be a general purpose computing device (e.g., a personal computer, personal digital assistant (PDA), cell phone, and so forth) or may be a computing device dedicated to programming IMD 60. Programmer 24 may be one of a clinician programmer or a patient programmer in some examples, i.e., the programmer may be configured for use depending on the intended user. A clinician programmer may include more functionality than the patient programmer. For example, a clinician programmer may include a more featured user interface that allows a clinician to download usage and status information from IMD 60, and allows the clinician to control aspects of IMD 60 not accessible by a patient programmer example of programmer 24.

A user (e.g., a clinician, patient 14 or a patient caregiver) may interact with processor 90 through user interface 94. User interface 94 may include a display, such as a liquid crystal display (LCD), light-emitting diode (LED) display, or other screen, to present information related to stimulation therapy, and buttons or a pad to provide input to programmer 24. Buttons may include an on/off switch, plus and minus buttons to zoom in or out or navigate through options, a select button to pick or store an input, and pointing device, e.g. a mouse, trackball, or stylus. Other input devices may be a wheel to scroll through options or a touch pad to move a pointing device on the display. In some examples, the display may be a touch screen that enables the user to select options directly from the display screen.

Processor 90 processes instructions from memory 92 and may store user input received through user interface 94 into the memory when appropriate for the current therapy. In addition, processor 90 provides and supports any of the functionality described in this disclosure with respect to each example of user interface 94. Processor 90 may comprise any one or more of a microprocessor, DSP, ASIC, FPGA, or other digital logic circuitry, and the functions attributed to processor 90 in this disclosure may be embodied as software, firmware, hardware or any combination thereof.

Memory 92 may include any one or more of a RAM, ROM, EEPROM, flash memory, or the like. Memory 92 may include instructions for operating user interface 94, telemetry module 96 and managing power source 98. Memory 92 may store program instructions that, when executed by processor 90, cause the processor and programmer 24 to provide the functionality ascribed to them in this disclosure. Memory 92 also includes instructions for generating therapy programs. In addition, in some examples, memory 92 stores mechanical stress information generated by mechanical stress sensor 70 (FIG. 4) of IMD 60 and transmitted to programmer 24 via the respective telemetry modules 68, 96 of IMD 60 and programmer 24.

Wireless telemetry in programmer 24 may be accomplished by RF communication or proximal inductive interaction of programmer 24 with IMD 60. This wireless communication is possible through the use of telemetry module 96. Accordingly, telemetry module 96 may include circuitry known in the art for such communication.

Power source 98 delivers operating power to the components of programmer 24. Power source 98 may include a battery and a power generation circuit to produce the operating power. In some examples, the battery may be rechargeable to allow extended operation. Recharging may be accomplished through proximal inductive interaction, or electrical contact with circuitry of a base or recharging station. In other examples, primary batteries may be used. In addition, programmer 24 may be directly coupled to an alternating current source, such would be the case with some computing devices, such as personal computers.

As previously indicated, in some examples, processor 62 (FIG. 4) of IMD 60 may store mechanical stress information 82 in memory 64 of IMD 60, where the mechanical stress information 82 can include the signal generated by sensor 70 or one or more values derived from the signal. For example, mechanical stress information 82 can include the amplitude (e.g., mean, instantaneous, minimum or maximum amplitude) of the signal generated by sensor 70, a resistance or a change in resistance of sensor 70 if sensor 70 indicates a mechanical load by changing resistance, a gross mechanical load or stress value or a change in mechanical load or stress value determined based on the signal generated by sensor 70, and the like. In addition to or instead of storing mechanical stress information 82 in memory 64 of IMD 60, processor 62 of IMD 60 may control telemetry module 68 to transmit the mechanical stress information to programmer 24 or another external device. Programmer 24 may store the information in memory 92 for later retrieval and analysis by a clinician or another user. In addition, IMD 60 or programmer 24 may transmit the mechanical stress information 82 to another device.

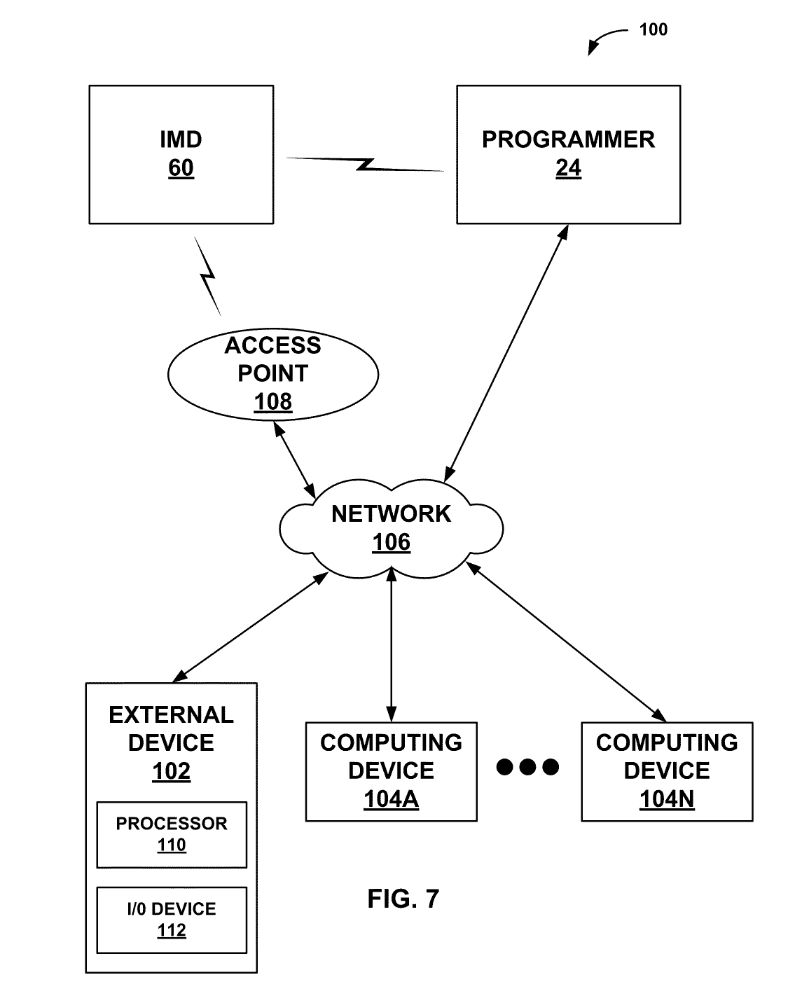

FIG. 7 is a block diagram illustrating a system 100 that includes an external device 132, such as a server, and one or more computing devices 104A-104N that are coupled to IMD 60 and programmer 24 via a network 106, according to one example. In this example, IMD 60 uses its telemetry module 68 (FIG. 4) to communicate with programmer 24 via a first wireless connection, and to communicate with an access point 108 via a second wireless connection. In the example of FIG. 1, access point 108, programmer 24, external device 102, and computing devices 104A-104N are interconnected, and able to communicate with each other, through network 106. In some cases, one or more of access point 108, programmer 24, external device 102, and computing devices 104A-104N may be coupled to network 106 through one or more wireless connections. IMD 60, programmer 24, external device 102, and computing devices 104A-104N may each comprise one or more processors, such as one or more microprocessors, DSPs, ASICs, FPGAs, programmable logic circuitry, or the like, that may perform various functions and operations, such as those described in this disclosure.

Access point 108 may comprise a device that connects to network 106 via any of a variety of connections, such as telephone dial-up, digital subscriber line (DSL), or cable modem connections. In other examples, access point 108 may be coupled to network 106 through different forms of connections, including wired or wireless connections. In some examples, access point 108 may communicate with programmer 24 and/or IMD 60. Access point 108 may be co-located with patient 14 (e.g., within the same room or within the same site as patient 14) or may be remotely located from patient 14. For example, access point 108 may be a home monitor that is located in the patient's home or is portable for carrying with patient 14.

During operation, IMD 60 may collect, measure, and store various forms of diagnostic data. For example, as described previously, IMD 60 may collect mechanical load information 82 that indicates a mechanical load exerted on IMD 60 and sensed by sensor 70 (FIG. 4) that is mechanically connected to housing 76 or connector 78 of IMD 60 or a component within housing 76 or connector 78. In certain cases, IMD 60 may directly analyze collected diagnostic data and generate any corresponding reports or alerts. In some cases, however, IMD 60 may send diagnostic data to programmer 24, access point 108, and/or external device 102, either wirelessly or via access point 108 and network 106, for remote processing and analysis.

For example, IMD 60 may send programmer 24 collected mechanical stress information 82, which is then analyzed by programmer 24. Programmer 24 may generate reports or alerts after analyzing the mechanical stress information 82 and determining that there may be a possible condition with IMD 60 based on the mechanical stresses sensed by sensor 70. As previously indicated, a mechanical stress is indicative of an average amount of force exerted per unit area of IMD 60. As another example, IMD 60 may send the mechanical stress information 82 to programmer 24, which may take further steps to determine whether there may be a possible condition with IMD 60 (e.g., a functional component within housing 76).

In some cases, IMD 60 and/or programmer 24 may combine all of the mechanical stress information into a single displayable system integrity report, which may be displayed on programmer 24. The system integrity report can include diagnostic information concerning the integrity of IMD 60 and its components. A clinician or other trained professional may review and/or annotate the system integrity report, and possibly identify any IMD integrity conditions, e.g., due to excessive and/or frequent stresses.

In another example, IMD 60 may provide external device 102 with collected mechanical stress information via access point 108 and network 106. External device 102 includes one or more processors 110. In some cases, external device 102 may request such data, and in some cases, IMD 66 may automatically or periodically provide such data to external device 102. Upon receipt of the diagnostic data via input/output device 112, external device 102 is capable of analyzing the data and generating reports or alerts upon determination that there may be a possible condition with IMD 60. For example, a mechanical joint between a circuit component and a circuit board within outer housing 76 of IMD 60 may experience a condition related to a fracture.

In one example, external device 102 may combine the diagnostic data into a system integrity report. One or more of computing devices 104A-104N may access the report through network 106 and display the report to users of computing devices 104A-104N. In some cases, external device 102 may automatically send the report via input/output device 202 to one or more of computing devices 104A-104N as an alert, such as an audio or visual alert. In some cases, external device 102 may send the report to another device, such as programmer 24, either automatically or upon request. In some cases, external device 102 may display the report to a user via input/output device 112.

In one example, external device 102 may comprise a secure storage site for mechanical stress information and, in some cases, other diagnostic information that has been collected from IMD 60 and/or programmer 24. In this embodiment, network 106 may comprise an Internet network, and trained professionals, such as clinicians, may use computing devices 104A-104N to securely access stored diagnostic data on external device 102. For example, the trained professionals may need to enter usernames and passwords to access the stored information on external device 102. In one embodiment, external device 102 may be a CareLink server provided by Medtronic, Inc., of Minneapolis, Minn.

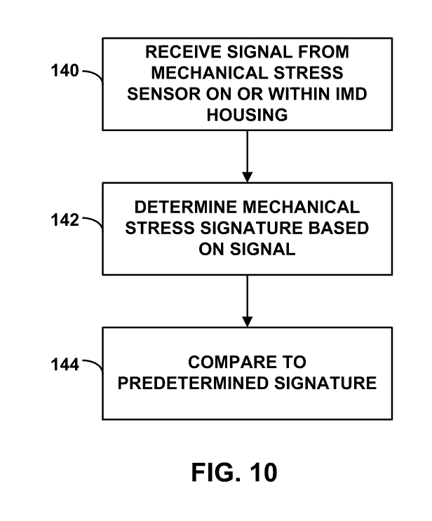

FIG. 8 is a flow diagram of an example technique with which a device may determine whether a mechanical stress sensed by sensor 70 of IMD 60 is indicative of a stress that may affect the operation of IMD 60. The mechanical stress may be generated by an anatomically induced mechanical load or a mechanical load attributable to an external source prior to, during or after implantation in patient 14. The example shown in FIG. 8 is primarily described with respect to monitoring in vivo mechanical loads, e.g., after implantation of IMD 60 in patient 14. The technique shown in FIG. 8 may also be used to determine whether a mechanical load exerted on IMD 60 is undesirable, e.g., because it may cause a mechanical stress within IMD 60 that may affect the operation of IMD 60. While the technique shown in FIG. 8, as well as FIGS. 9 and 10 are described with respect to processor 62 of IMD 60 (FIG. 4), in other examples, processor 90 (FIG. 6) of programmer 24 or a processor of another device may perform any part of the technique shown in FIG. 8.

Processor 62 receives a signal generated by implanted mechanical stress sensor 70 (120), where at least one characteristic of the signal changes as a function of the mechanical load exerted on IMD 60. Processor 62 determines a mechanical stress that generated within IMD 60 based on the signal (122), where the mechanical stress may be a transient or a cumulative mechanical stress (e.g., an average or median stress value over a period of time). For example, processor 62 may determine a change in the mechanical stress or a value indicative of the total mechanical stress based on at least one time domain characteristic (e.g., an amplitude) of the signal generated by sensor 70. In some examples, the time domain characteristic is a mean, median or peak characteristic of the signal over a sample period of time. The change in the mechanical stress or the gross mechanical stress value may be correlated to a particular load value. Thus, in some examples, processor 62 determines an in vivo mechanical load exerted on IMD 60 based on the signal generated by sensor 70.

Processor 62 determines whether the mechanical stress sensed by sensor 70 is greater than or equal to a predetermined threshold stress value (124), which may be stored in memory 64 of IMD 60 or a memory of another device (e.g., programmer 24). Alternatively, processor 62 may merely determine whether the in vivo mechanical load exerted on IMD 60 is greater than or equal to a predetermined threshold load value. The predetermined load value can be, for example, a transient or cumulative stress value that potentially indicates stress fatigue of housing 76, connector block 78, therapy delivery member 74 or other component to which sensor 70 is mechanically coupled.

In either example, processor 62 may select a predetermined threshold value from memory 64. In some examples, memory 64 stores a plurality of threshold values. For example, processor 62 may select a threshold value that is specifically associated with a particular patient posture state or activity level, which may be determined, e.g., based on a signal generated by an accelerometer that is separate from mechanical stress sensor 70. The interface between IMD 60 and adjacent tissue may change depending on the patient posture, and, as a result, the tissue may exert different magnitudes of force on IMD 60 based on the patient posture. Thus, in some cases, posture-specific threshold values may be useful for monitoring the in vivo stresses exerted on IMD 60. In other examples, however, processor 62 monitors the mechanical load exerted on IMD 60 irrespective of the posture or activity level of patient 14 by using a predetermined threshold value that does not change based on a determined patient posture state or activity level.

In one example, processor 62 compares one or more of the average, median, peak or cumulative peak amplitudes of the signal generated by sensor 70 to the applicable threshold value. Different threshold values may be stored for each of the applicable types of values (e.g., one or more of the one or more of the average, median, peak or cumulative peak amplitudes). If the one or more of the average, median, peak or cumulative peak amplitudes of the signal is not greater than or equal to the predetermined threshold value (124), processor 62 continues to monitor the mechanical stress by receiving the signal from sensor (70), determining the in vivo mechanical stress of IMD 60 (72), and comparing the in vivo mechanical stress to the predetermined threshold value (124). Processor 62 may continue monitoring the mechanical load exerted on IMD 60 using the technique shown in FIG. 8 at any suitable frequency, such as a frequency of about 1 Hertz (Hz) to about 500 Hz, such as about 10 Hz to about 100 Hz. However, other sampling rates may also be suitable depending upon the application for which the signal for sensor 70 is used.

If one or more of the average, median, peak or cumulative peak amplitudes of the signal are greater than or equal to an applicable predetermined threshold stress value (124), processor 62 generates a stress indication (126). The stress indication can be, for example, a value, flag, signal or any other marker that can be stored in memory 64 of IMD 60 or transmitted to another device (e.g., external programmer 24) that indicates that a transient or a cumulative mechanical stress exceeding a desirable level has been detected. In some examples, IMD 60 transmits the stress indication to an external device using system 100 shown in FIG. 7. In addition, in some examples, processor 62 may generate a notification to patient 14, a patient caregiver or a clinician upon the generation of the stress indication. For example, processor 62 may cause IMD 60 to vibrate (e.g., in a particular pattern as indicated by the intensity or timing of the vibration) or processor 62 may transmit a signal to programmer 24 or another external device. The programmer or other external device may generate an auditory, visual or somatosensory alert to notify patient 14 or the patient caretaker that the stress indication was generated.

The technique shown in FIG. 8 may also be used to monitor the loads exerted on IMD 60 and/or therapy delivery member 74 (in examples in which a sensor 70 is coupled to member 74) during implantation of IMD 60 and/or therapy delivery member 74 in patient 14. This may be useful for monitoring the implant techniques implemented by a clinician. As discussed above, the loads that are exerted on IMD 60 and therapy delivery element 74 during implantation in patient 14 may be attributable to various sources, such as the use of hemostats, manual bending of therapy delivery element 74 by the clinician, as well as tensile or compressive forces resulting from the handling of IMD 60 and/or therapy delivery element 74. If the implant technique results in a mechanical load that is greater than or equal to a predetermined threshold value, processor 62 of IMD 60 (or a processor of another device, such as programmer 24) may generate a stress indication and, in some cases, generate a notification to the clinician.

As previously indicated, in some examples, a patient parameter may be determined based on the mechanical stress information generated by sensor 70 of IMD 60 when IMD 60 is implanted within patient 14. In some examples, the determined patient parameter may be used to control therapy delivery to patient 14. In other examples, the patient parameter does not affect therapy delivery. In either example, the determined patient parameter may be stored in memory 64 of IMD 60 or a memory of another device.

FIG. 9 is a flow diagram of an example technique for controlling therapy delivery to patient 14 based on a signal generated by mechanical stress sensor 70. In accordance with the technique shown in FIG. 9, processor 62 receives a signal generated by implanted mechanical stress sensor (120) and determines a patient parameter based on the signal (130).

In some examples, the patient parameter comprises an occurrence of a cardiac contraction (e.g., physiologically significant cardiac contraction that indicates heart 12 (FIG. 1) is contracting to provide patient 14 with sufficient cardiac output to meet the physiological demands of the patient's body) or the absence of the cardiac contraction within a sample period of time. Processor 62 may determine, for example, whether a peak mechanical load exerted on IMD 60 within a predetermined duration of time is greater than or equal to a predetermined threshold value that is indicative of an occurrence of a physiologically significant cardiac contraction.

The mechanism by which a signal generated by sensor 70 is indicative of a cardiac contraction may change depending on a proximity of IMD 60 to heart 12. In some examples, an occurrence of a physiologically significant cardiac contraction results in an increased blood flow through tissue of patient 14 (e.g., an increased tissue perfusion value), which may result in an increased pressure exerted on IMD 60 (or therapy delivery member 74 if sensor 70 is mechanically coupled to member 74). In other examples, sensor 70 may sense the movement of heart 12 resulting from the cardiac contraction. The movement of heart 12 may result in a change in pressure exerted on IMD 60 (or therapy delivery member 74), which may be sensed by sensor 60.

The cycle of mechanical loads indicated by the signal generated by sensor 70 may be indicative of the timing of the cardiac contractions, which may be indicative of a heart rate of patient 14. Thus, in some examples, processor 62 may determine the timing between the peak mechanical loads sensed by sensor 70 to determine a heart rate of patient 14. Other types of patient parameters may be determined based on the signal from sensor 70. For example, as indicated above, the patient parameter may be a force measurement at a particular joint of patient 14 or pressure information indicative of pressure ulcer formation conditions.

In some examples, processor 62 stores the determined patient parameter in memory 64 or transmits the determined patient parameter to another device (e.g., programmer 24) for storage. In addition, in the technique shown in FIG. 9, processor 62 controls therapy delivery module 66 (FIG. 4) based on the determined patient parameter (132). For example, if the determined patient parameter is the absence of a physiologically significant cardiac contraction within a predetermined range of time in which the cardiac contraction is expected, and therapy delivery module 66 is configured to deliver electrical stimulation therapy to heart 12 (e.g., as shown in FIG. 1), processor 62 may control therapy delivery module 66 to deliver a defibrillation shock to heart 12 upon detecting the patient parameter indicative of the absence of a physiologically significant cardiac contraction.

As another example, if the determined patient parameter is a heart rate, processor 62 may determine whether the heart rate of patient 14 indicates delivery of cardiac rhythm therapy to heart 12 is desirable. For example, if the heart rate is relatively slow, thereby indicating insufficient cardiac activity, processor 62 may control therapy delivery module 66 to deliver pacing therapy (e.g., cardiac resynchronization therapy) to heart 12. In other examples, if therapy delivery module 66 delivers pacing therapy to heart 12, processor 62 may control the pacing rate based on the heart rate determined based on the cycle of mechanical loads sensed by mechanical stress sensor 70.

The signal generated by mechanical stress information 70 may be used to track the mechanical loads exerted on IMD 60. FIG. 10 is a flow diagram of an example technique for determining the mechanical loads exerted on IMD 60 over time. The technique shown in FIG. 10 may be used at any time, such as while IMD 60 is implanted in patient 14, during implantation of IMD 60 and/or therapy delivery member 74 in patient 14, or during manufacturing, storage, shipping, or other handling of IMD 60 prior to or after implantation in patient 14.

Processor 62 receives a signal generated by sensor 70 (140) and determines a mechanical stress signature based on the signal (142). The mechanical stress signature may be, for example, a pattern in the signal waveform over time. As mechanical loads are periodically applied to IMD 60 and removed from IMD 60, the amplitude of the signal generated by sensor 70 may change, thereby changing the waveform shape. The pattern in the signal waveform is indicative of the load application timing and magnitude over time.

In some cases, the manufacturing, storage, shipping, and/or other handling of IMD 60 during the normal course of the life of IMD 60 may be known to have a predetermined signature. The predetermined signature may be determined based on the pattern in mechanical stress indicated by a mechanical stress sensor connected to the IMD when the IMD is known to have undergone the expected manufacturing, storage, shipping, and/or other handling process. Similarly, the implantation of IMD 60 and therapy delivery member 74 may be known to have a predetermined signature. The predetermined signature may be determined based on the pattern in mechanical stress resulting from the handling and manipulation of IMD 60 and/or therapy delivery member 74 in patient 14.

Processor 62 of IMD 60 may compare the mechanical stress signature to the predetermined signature (144), which may be stored in memory 64 or programmer 24. The comparison may indicate whether the particular IMD 60 for which the stress signature is determined has been exposed to any unexpected or otherwise abnormal stresses. In this way, the mechanical stress signature that is determined based on sensor 70 may be used for quality control purposes.

Processor 62 may compare the mechanical stress signature to the predetermined signature (144) by correlating the mechanical stress signature to the predetermined signature. For example, processor 62 may correlate an amplitude waveform of the signal generated by sensor 70 in the time domain or frequency domain with a template signal that is indicative of the mechanical stress signature. Processor 62 may compare a slope of the waveform of the mechanical stress signature or timing between inflection points or other critical points in the pattern of the mechanical stress signature over time to the mechanical stress signature template.

A substantial correlation between the mechanical stress signature and the predetermined mechanical stress signature template may indicate that IMD 60 was subjected to the expected mechanical loads. In some examples, a 100% match between the determined mechanical stress signature and the predetermined stress signature (e.g., as determined by a match in the amplitude values over time) may not be necessary to determine that the mechanical stress signature substantially correlates to the predetermined signature. For example, a match rate of about 75% to about 100% match between the waveform patterns of the mechanical stress signature and predetermined signature template may indicate that the mechanical stress signature substantially correlates to the predetermined signature.

If the comparison indicates that the determined mechanical stress signature does not substantially correlate to the predetermined stress signature, processor 62 may determine that that IMD 60 was subjected to mechanical loads that differed from the expected mechanical loads (as indicated by the predetermined signature). Processor 62 may then generate a deviation indication. The deviation indication may be a flag, value signal or any other marker that can be stored in memory 64 of IMD 60 or transmitted to another device (e.g., external programmer 24) that indicates that IMD 60 was subject to mechanical loads that deviated (e.g., differed) from the expected mechanical loads. In some examples, processor 62 transmits the indication to an external device using system 100 shown in FIG. 7.

In some examples of the technique shown in FIG. 10, a device other than IMD 60 receives the signal generated by sensor 70 (140) during at least one of the manufacturing, storage, shipping or other handling of IMD 60 and determines the mechanical stress signature based on the signal (142). The device may compare the determined mechanical stress signature to the predetermined signature to determine whether the particular IMD 60 may have been subjected to mechanical loads that differ from the expected mechanical loads.

In some cases, a medical device is implanted in a patient proximate a muscle. For example, a medical device can be implanted in a submuscular location within a patient, e.g., under a muscle in a deep direction (or deep position) relative to the epidermis of the patient. In cases in which an IMD is implanted in a submuscular location or otherwise proximate a muscle, the muscle can exert a compressive force on the IMD. This compressive force can also be referred to as a normal force because the muscle exerts a force in a direction substantially orthogonal to a direction (or line) in which the muscle contracts. As an example of the compressive force, when a medical device is implanted proximate the pectoralis major muscle of a human patient, the muscle can create a compressive mechanical loading on the outer housing of an IMD that is implanted predominantly transversely to the line of action of the pectoralis major muscle. While the mechanical stress sensors described above can be useful for determining the compressive force exerted by a muscle on an IMD, it can also be useful to determine the compressive force based on the parameters of the muscle and a force exerted by the muscle substantially along a direction of a line of action (e.g., contraction) of the muscle (generally referred to in this disclosure as an in-line muscle force).

A transfer function that indicates a relationship between an in-line muscle force (e.g., a force induced by a muscle substantially along a direction in which the muscle contracts) and a force substantially normal to the direction in which the in-line muscle force is induced can be used to determine the compressive forces exerted on the IMD. The transfer function can be used in addition to or instead of a mechanical stress sensor mechanically coupled to at least one of a housing of an IMD or a component within the housing of the IMD.

An example transfer function that has been determined to indicate the relationship between muscle parameters (e.g., dimensions), in-line muscle forces, and normal muscle forces with relatively high accuracy is as follows:

Normal Force=77.6+0.264*In-Line Force−1.44*Muscle Length−18.2*Muscle Width−139*Muscle Thickness+27.5*Physiologic Cross-sectional Area of Muscle−0.139*Measured Volume+0.110*Muscle Mass+0.0326 (Equation 1)

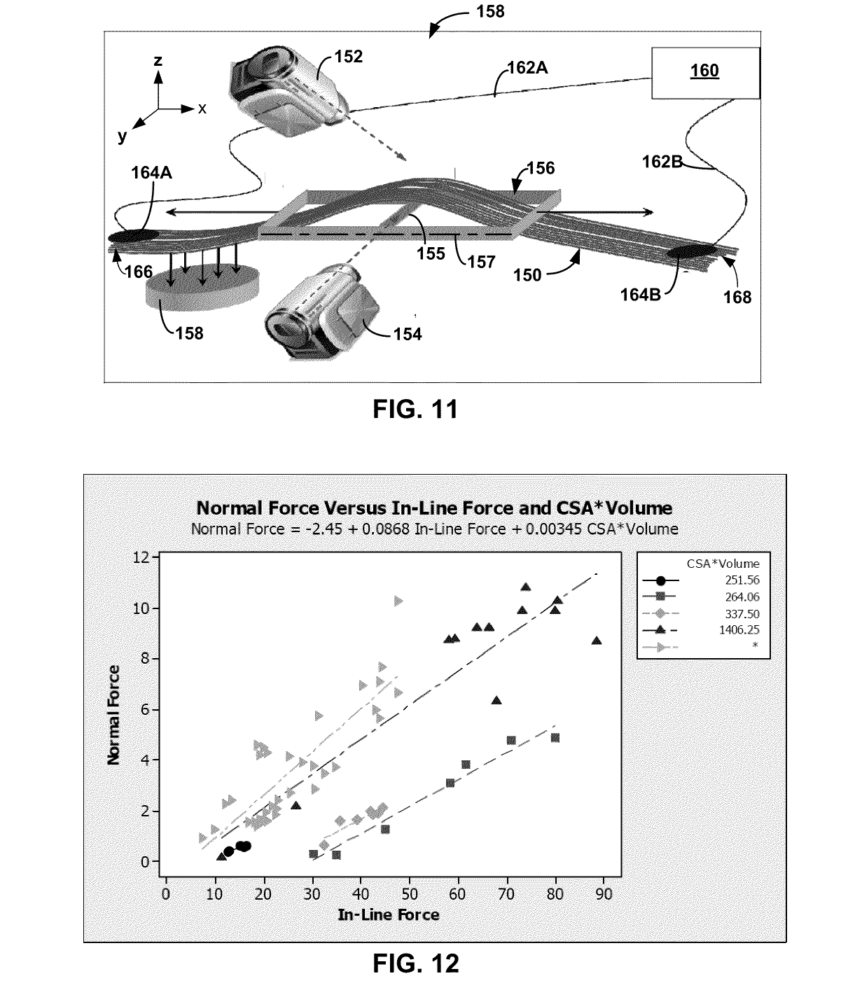

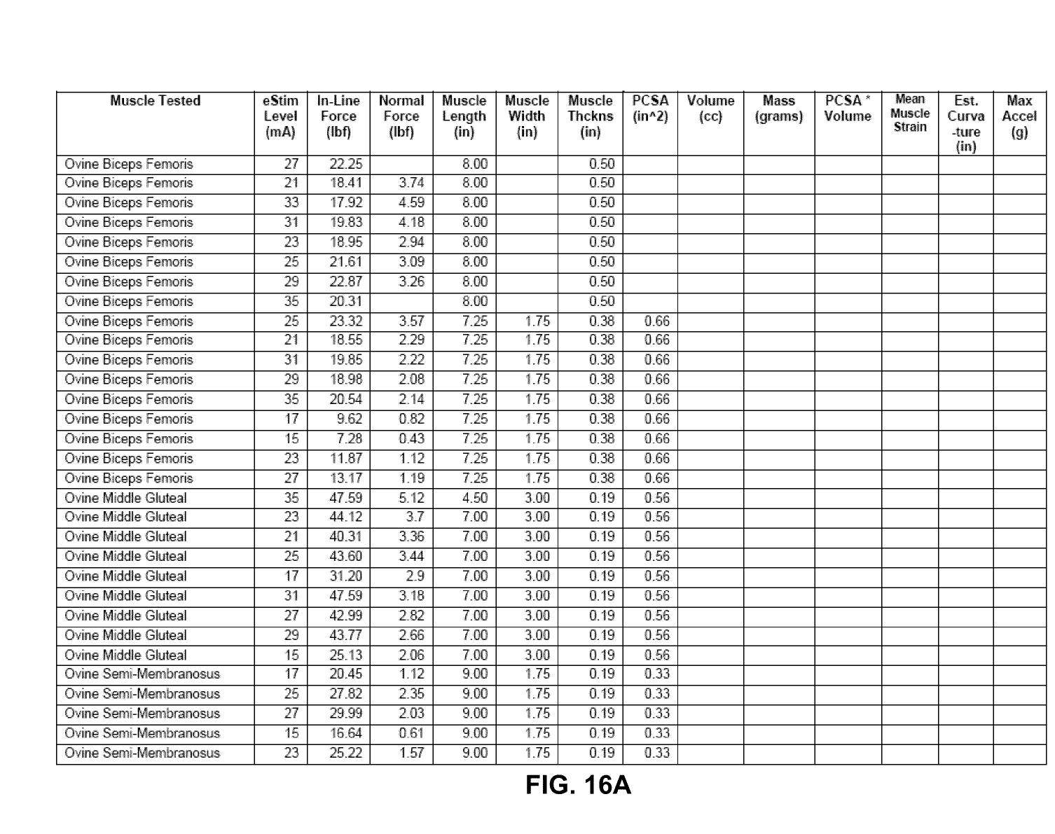

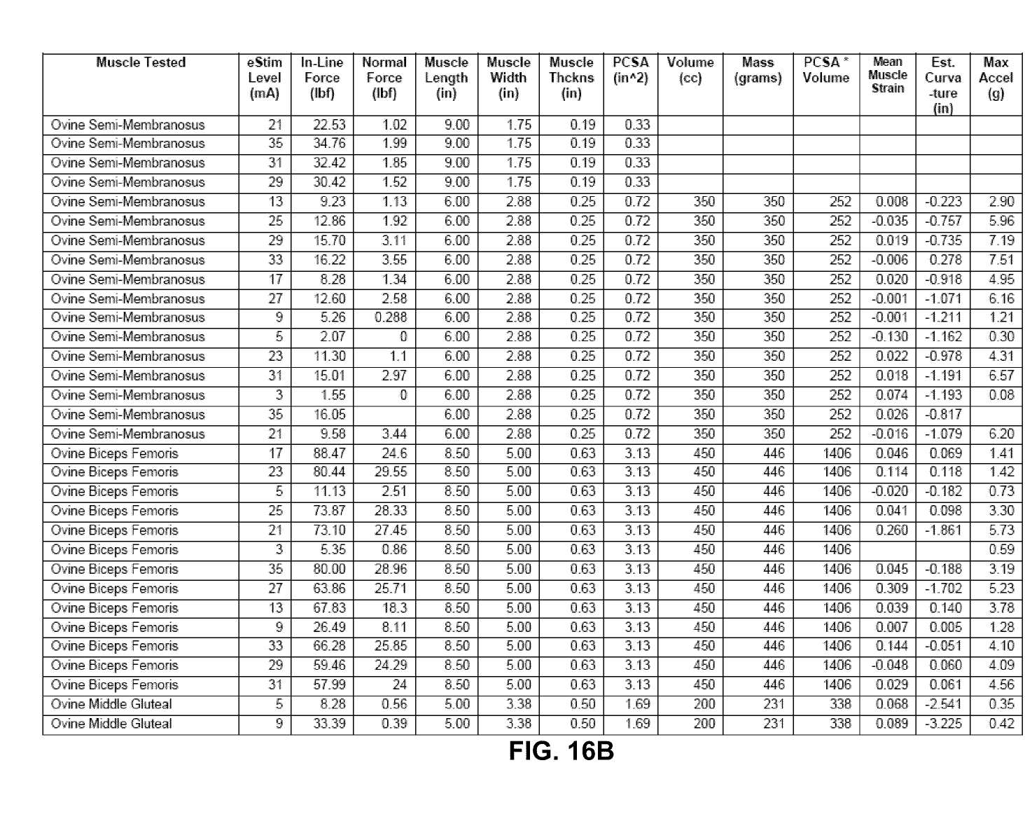

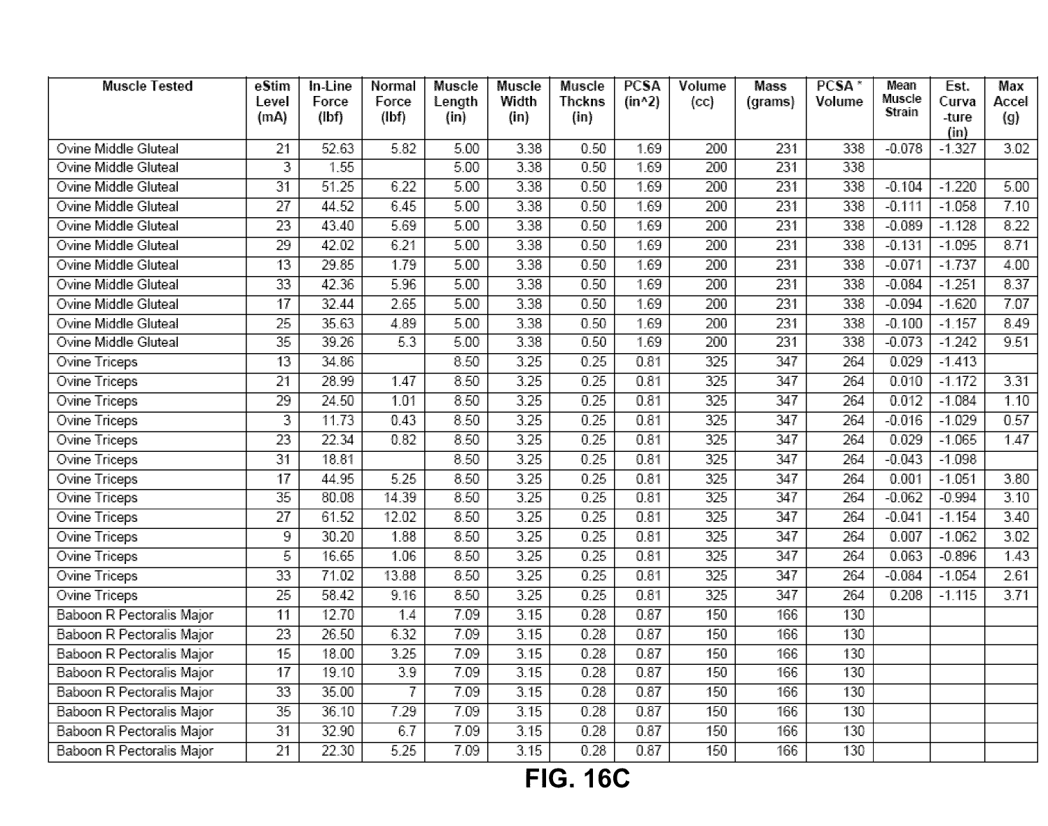

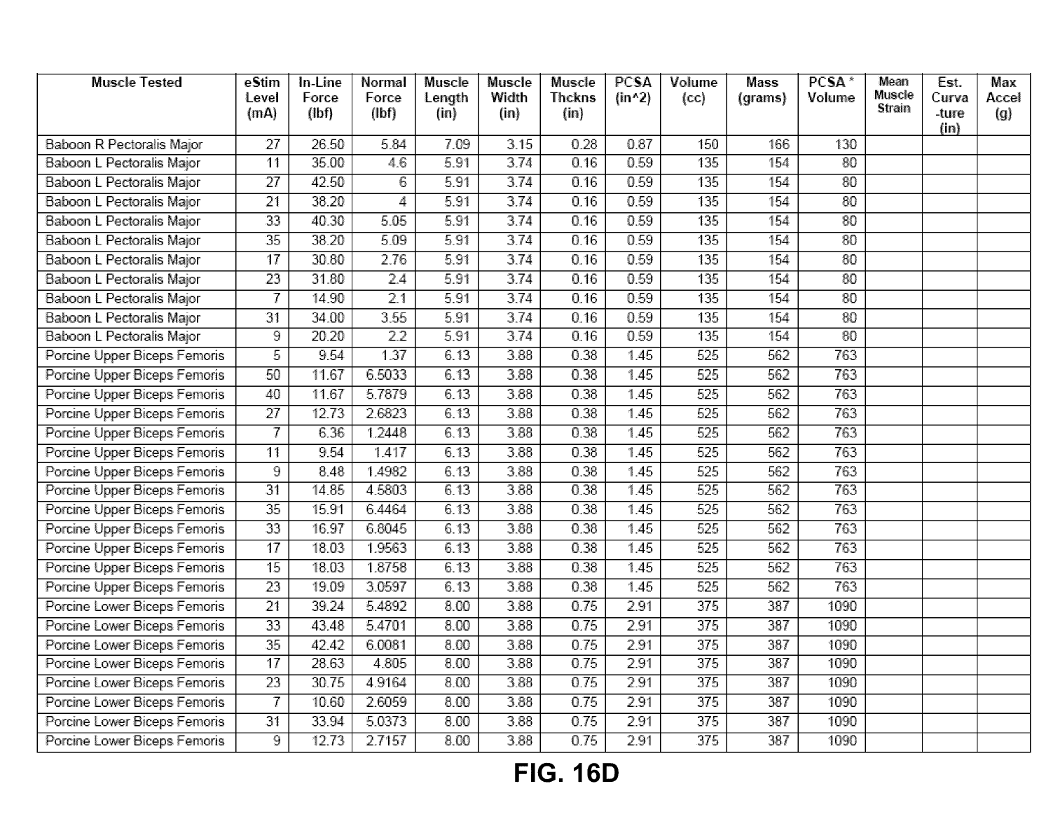

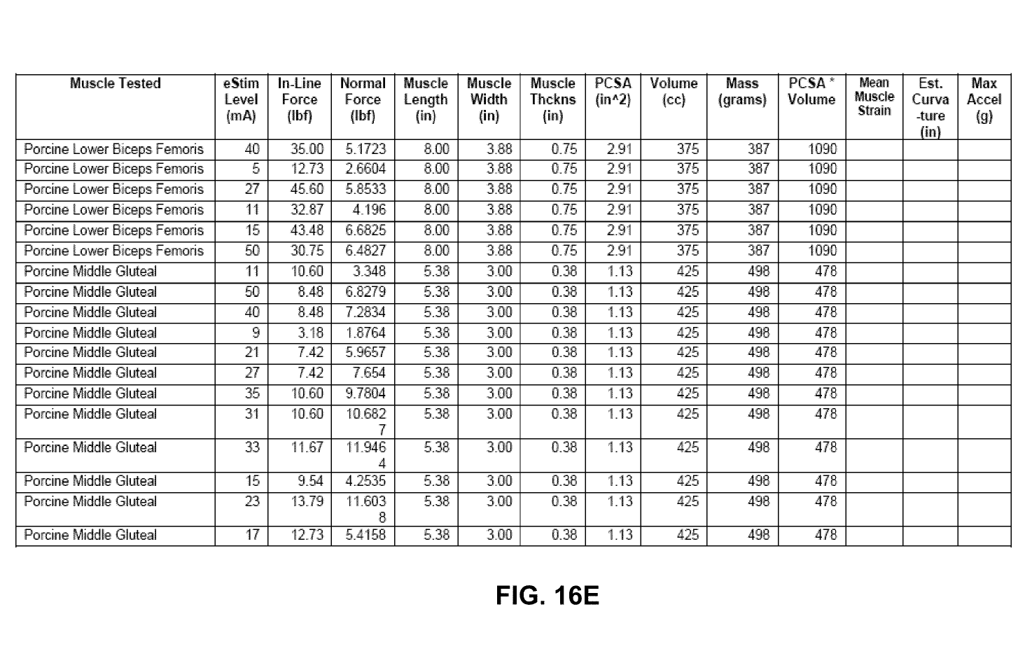

The * symbol in the above equation indicates the mathematical multiplication operation. The variables of the transfer function (Equation 1) may vary depending upon the data set on which the transfer function is determined. An example technique for determining the transfer function is described below with reference to FIGS. 11-14. In Equation 1, the unit of the in-line force is pounds, the unit of the muscle length, width, and thickness is inches, the unit of the physiologic cross-sectional area of muscle is square inches, the volume of the muscle is centimeters squared, and the mass of the muscle is grams. The data for ovine, porcine, and non-human primate (and, in particular, Chacma baboon) subjects analyzed to arrive at Equation 1 is shown in FIGS. 16A-16E.

There is a strong correlation between normal force exerted by a muscle and an in-line force generated by the muscle during contraction and muscle parameters. This strong correlation indicates that the in-line force and muscle parameters can be used to estimate normal force exerted by a muscle, which can be useful for determining in vivo mechanical loading conditions on an implanted device. In examples in which the muscle parameters include muscle length, muscle width, muscle thickness, physiologic cross-sectional area of the muscle, muscle volume, and muscle mass, the transfer function (Equation 1) indicates this relationship between normal force and in-line force. The transfer function defines a relationship between parameters that can be measured relatively easily and noninvasively (e.g., in-line muscle forces and muscle parameters) and the mechanical load exerted on the medical device from the forces generated by the muscle.

The in-line muscle forces can be determined using any suitable technique. In some examples, the Hill Muscle Model is used to determine the in-line muscle forces. The Hill Muscle Model relates to determining the production of force by a muscle along its line of action. The Hill Muscle Model establishes that for a given sustained level of neural excitation (e.g., as indicated by an EMG), a sudden change in force (or length) would result in a nearly instantaneous change in length (or force). In-line muscle forces can be determined (e.g., predicted or estimated) using non-invasively collected muscle parameters, such as muscle length, and an electromyogram (EMG), which indicates the electrical potential generated by a muscle. An EMG can be generated using external electrodes (e.g., surface electrodes) attached to an epidermis of a patient proximate the muscle or muscle group of interest, percutaneous electrodes (e.g., needle electrodes) or fully implanted electrodes.

The geometric muscle parameters (i.e., length, width, thickness, cross-sectional area and volume) can be determined using any suitable technique. A length of a muscle can be the distance from the muscle origin to the point of insertion, where the origin is the point at which the muscle attaches to a structure, such as a bone, tendon or another muscle, and the point of insertion is at the opposite end of the muscle from the origin. The thickness of the muscle can be the thickness at the portion of the muscle proximate the implanted device, the greatest thickness of the muscle, the minimum thickness of the muscle, an average thickness of the muscle, and the thickness at another portion of the muscle. Similarly, the width of the muscle can be the width at the portion of the muscle proximate the implanted device, the greatest width of the muscle, the minimum width of the muscle, or an average width of the muscle, or the width at another portion of the muscle. The physiologic cross-sectional area (PCSA) can be determined based on the following equation:

$$P C S A = \frac{{V}_{m}}{{L}_{m}} \cdot \frac{{L}_{f}}{{L}_{f , opt}} \qquad ( Equation 2 )$$

In Equation 2, Vₘ is the volume of the muscle, Lₘ is the length of the muscle, L_{f} is the length of the muscle fiber, and L_{f,opt} is the optimal muscle fiber length. The physiologic cross-sectional area was included as part of the study because it scales proportionally with a maximum isometric force of a muscle at an optimal muscle fiber length (which is assumed to be similar to the muscle fiber length for purposes of this example). Muscle fibers make up the overall muscle and are arranged to effectively actuate a joint. In Equation 2, the length of the muscle fiber L_{f} is the specifically involved length of the muscle tissue over the implanted system (e.g., the IMD or, in the example of FIG. 11, force sensor 158) when the muscle is relaxed (e.g., not contracting or contracting a minimal amount). The optimal length L_{f,opt} is the length of the muscle at which, during exertion, the muscle is producing the highest tension. For the example shown in FIG. 11, the physiologic cross-sectional area (PCSA) determination was more simplified compared to Equation 2 because the cross sectional area of the muscle in the section that was approximately over the center of the force sensor (force sensor 158 shown in FIG. 11) was measured when the muscle (muscle 150 shown in FIG. 11) was in a relaxed state. Equation 2 is more for a Hill Model estimation and is valid for such. The simplified physiologic cross-sectional area (PCSA) measurement method was sufficient for the example shown in FIG. 11 given the high predictability resulting from the transfer function (Equation 1).

Geometric muscle parameters (i.e., length, width, thickness, cross-sectional area and volume) can be determined externally using any suitable technique. In some examples, a magnetic resonance image (MRI) or another suitable type of medical image (e.g., X-ray) of the muscle can be captured and the muscle parameters can be estimated based on the medical image. In addition to or instead of the medical image, the muscle parameters can be estimated using other measurable parameters of the patient, such as height, weight, body mass index or similar parameters. In some examples, the volume of the muscle can be estimated based on an average volume of the muscle for a plurality of patients.

The in-line muscle forces can be determined noninvasively, e.g., with an external, rather than an implanted device, although an implanted device can also be used. Moreover, the muscle parameters can also be measured noninvasively. Therefore, the transfer function (e.g., Equation 1) is useful for predicting compressive loading on an IMD from muscles proximate the IMD without requiring the implantation of a load-indicating device in a patient. As discussed above, determining in vivo mechanical loading conditions for an IMD resulting from internal forces (e.g., generated by a muscle) can be useful for various purposes, such as, but not limited to, selecting implant parameters, such as a design of the IMD (e.g., selecting a shape, thickness, or material for the IMD housing), a type of IMD to be implanted in the patient, determining expected implant conditions for an IMD for a particular patient or a class of patients, determining reliability of a particular IMD design (e.g., housing thickness, geometry or material), or even determining physiological parameters of a patients. Examples of types of IMDs include different design models, which can have different sizes, geometric configurations, volumes, masses, and the like.

In addition, determining the mechanical loading conditions (e.g., compressive forces) to which the IMD may be exposed can be useful for selecting an implant site for the IMD within a patient. For example, for each of the implant sites that are being considered, a clinician can identify the muscles that may exert forces on an IMD, and determine the muscle parameters for the identified muscles. Example implant sites for an implantable pacemaker or implantable cardioverter defibrillator include, but are not limited to, a retro mammary implant site, abdominal implant site, and pectoral implant site. With the aid of the transfer function (e.g., Equation 1 described above) that indicates the relationship between in-line muscle forces and compressive muscle forces, the clinician, alone or with the aid of a computing device, can determine the mechanical loads that may be exerted on an IMD at each of the implant sites.

In some examples, processor 90 of programmer 24 (FIG. 6) or a processor of another device automatically determines the compressive force exerted by a particular muscle for a specific patient based on the in-line muscle force and muscle parameters. In some examples, the in-line muscle force and/or muscle parameters are automatically determined. For example, processor 90 of programmer 24 or another device can be electrically coupled (via a wired or wireless connection) to one or more sensors (e.g., a buckle transducer or external EMG) that determines the in-line muscle force, and processor 90 can receive input from the sensor from which the in-line muscle force can be determined. In other examples, a clinician can provide input (e.g., via user interface 94) that indicates the in-line muscle force and/or muscle parameters for a particular muscle, and processor 90 can determine the compressive force exerted by the muscle based on the inputted data.

Both the in-line forces exerted by a muscle and the geometrical parameters of a muscle or muscle group of interest can be determined externally (e.g., without an invasive surgical technique). Therefore, using the transfer function (e.g., Equation 1), the compressive muscle forces that may act on an IMD at a particular target tissue site can be determined with externally measured parameters. As a result, the clinician can evaluate the various implant sites for an IMD prior to implanting any device or at least prior to implanting the IMD in the patient.

The clinician can compare the implant sites based on the determined mechanical loads. In some cases, the clinician selects the implant site associated with the lowest mechanical load. However, the clinician can also balance the expected mechanical loads at a particular implant site with other considerations, such as, but not limited to, the invasiveness of the medical device implantation process for a respective implant site, the risk of infection for a respective implant site, discomfort to the patient when an IMD is implanted at the respective implant site, cosmetic considerations (e.g., visibility of the IMD at a particular implant site), and expected healing time for the respective implant site.

Studies were conducted on a large variety of muscles across a diverse group of nonhuman mammal subjects (selected to be approximately the same size as humans) to develop the relationship between the in-line muscle forces and compressive muscle forces. The transfer function was established based on these studies. The nonhuman mammal subjects included ovine, porcine, and non-human primate (and, in particular, Chacma baboon) subjects. A test device was implanted under (i.e., a deep site) a muscle of the nonhuman subject, whereby the test device did not include therapy delivery capabilities and was used as a sensorized stand-in for an IMD. The muscle by which the test device was implanted was selected to be similar in size and structure to a muscle by which an IMD would be implanted within a human subject. For example, in an ovine subject, test devices were implanted under each of a biceps femoris, a long-head of the triceps, and a semi-tendinosus. In a porcine subject, test devices were implanted under the bicep femoris, and middle gluteal. In a nonhuman primate, test devices were implanted under the left and right pectoralis major muscles.

FIG. 11 is a schematic diagram illustrating an example system 158 that was utilized to conduct the studies and determine which muscle parameters are useful for defining a transfer function that indicates the relationship between in-line muscle forces and compressive muscle forces. System 158 includes cameras 152, 154, buckle transducer 156, and force sensor 158. The technique utilized to conduct the study to determine the relationship between the transfer function that indicates the in-line muscle forces and compressive muscle forces is generally described with respect to FIG. 11.

In order to determine the in-line muscle force exerted by muscle 150 of a nonhuman subject, a clinician exposed muscle 150 of interest and measured the length, thickness, and width of muscle 150. Each muscle of the nonhuman subjects were measured in this manner to collect predetermined values for a common set of muscle parameters. Anatomical landmarks were used to measure each of the muscles of interest in a consistent manner. For example, with respect to the length of muscle 150, the length was measured from the muscle origin 166 to the point of insertion 168. Origin 166 of muscle 150 is the point at which muscle 150 attaches to a structure, such as a bone or another muscle, and point of insertion 168 is the end of muscle 150 opposite origin 166.

The thickness of muscle 150 was measured in a z-axis direction at cross-bar 155 of buckle sensor 156, which is described in further detail below. The width of muscle 150 was measured in a y-axis direction at cross-bar 155 of buckle sensor 156. The mass and volume of muscle 150 were determined using a scale and fluid displacement method, respectively. The pectoralis major muscles of the baboon subjects had an average mass of about 131.8 grams with a standard deviation of about 37.4 grams, and a volume of about 118.8 cubic centimeters with a standard deviation of about 34.7 cubic centimeters, resulting in a material density of about 1.115 grams per cubic centimeter (g/cm³) with a standard deviation of about 0.055 grams per cubic centimeter (g/cm³).

Electrical stimulator 160 was electrically coupled to electrodes 164A, 164B via respective leads 162A, 162B. Electrical stimulator 160 generates and delivers electrical stimulation to muscle 150 via electrodes 164A, 164B in order to induce contractions of muscle 150. The induced muscle contractions simulate movement of muscle 150 during normal activities undertaken by the subject. The current amplitude of the electrical stimulation delivered to induce muscle contractions was selected to be relatively nonlinear in order to avoid muscle fatigue. In the study on the baboons, the electrical stimulation was delivered as trains of electrical current pulses of constant, discrete pulse amplitudes of about 3 milliamps (mA), about 5 mA, about 7 mA, about 9 mA, about 11 mA, about 15 mA, about 17 mA, about 21 mA, about 23 mA, about 27 mA, about 31 mA, about 33 mA, and about 35 mA in a predetermined, randomized order. Each pulse train comprised about 2000 pulses with an individual pulse duration of about 53 microseconds and a pulse interval of about 203 microseconds. The full stimulation exposure duration was about 4 seconds. The pulse amplitude ranges, duration and randomization were selected to reach the full range of activation of muscle 150 and to minimize fatigue. The maximum forces sensed by each sensor 156, 158 were determined for each stimulation amplitude. Cameras 152, 154 were set up to view exposed portions of muscle 150 and were used to obtain visually recordings of the contractions of muscle 150 and verify the occurrence of muscle contractions.

Buckle sensor 156 (also referred to as a buckle transducer) includes two linear foil strain gauges (Model EA-DY-125BT-350, Vishay Micro Measurements Group of Malvern Pa.), which each generate a signal calibrated to in-line force, and, therefore, indicative of in-line force. Together, the strain gauges indicate a force in a direction substantially parallel to the direction in which the longitudinal axis 157 extends. Buckle sensor 156 has a closed rectangular frame of about 66 millimeters (mm) by about 100 mm, and a thickness (measured in the z-axis direction, where orthogonal x-y-z axes are shown in FIG. 11) of about 4 mm. Center bar 155 has a semi-circular cross-section (taken in the x-z plane) of about 2 mm. Center bar 155 was positioned such that the curvilinear surface of the semi-circular cross-section was facing muscle 150.

Force sensor 158 is similar to IMD 60 described with respect to FIG. 5, but includes a different distribution of the sensors 70. In particular, force sensor 158 has a foot print and approximate size of an implantable medical device and comprised a medical grade epoxy cast comprising six force load sensors (provided by Tekscan of Boston, Mass.). In contrast to IMD 60 of FIG. 5, the load sensors in housing 76 of force sensor 158 were distributed in two rows, where a row closest to connector block 78 included three load sensors and the row furthest from connector block 78 included two load sensors. Titanium cover plates were provided over each force sensor. Force sensor 158 has a volume of approximately 29 cubic centimeters and is approximately 64 mm by approximately 61 mm by approximately 11 mm.

Force sensor 158 includes a telemetry module that transmits information to a computing device via radio frequency (RF) communication signals, as well as a three-axis accelerometer (provided by Freescale Semiconductor of Tempe Ariz.), a processor, a clock, and a power source. The processor controlled the transmission of force readings by the force sensors to the computing device (a desktop computer) via the telemetry module. The load sensors of force sensor 158 were preconditioned for about four weeks with a mild static compression load and a 100% relative humidity at a temperature of about 37 degrees Celsius in order to simulate in vivo conditions and stabilize the load sensors. In addition, because the sensitivity of the load sensors changed over time due to, e.g., the varying conductance of the sensors, a calibration was performed within about four hours after explantation from the subject to provide a reference for data analysis.

For data analysis purposes, it was assumed that the compressive force was equally distributed across the force sensor 158 surface, such that the total compressive force F_{T} was determined based on the following equation:

$${F}_{T} = \frac{{A}_{IPM}}{{\sum }_{i = 1}^{6} {A}_{Si}} {\sum }_{i = 1}^{6} {F}_{Si} \qquad ( Equation 3 )$$

F_{Si} is the individual forces recorded by the six load sensors, A_{Si} is the surface areas of the titanium sensor cover plates, and A_{IPM} is the in-plane surface of force sensor 158 (e.g., the total area adjacent muscle 150).

Force sensor 158 was implanted under muscle 150 (i.e., in a deep direction, whereby the positive z-axis direction in FIG. 11 indicates a superficial direction and the negative z-axis direction indicates a deep direction; the orthogonal x-y-z axes are shown in FIG. 11 for ease of description of FIG. 11 only). Force sensor 158 was sutured in place 150 during the study on each nonhuman subject. Fibrous encapsulation was not permitted to form around force sensor 158 prior to implantation of buckle sensor 156 and implementation of cameras 152, 154 (i.e., prior to setting cameras 152, 154 up to view an exposed portion of muscle 150). In addition, to determine the in-line force exerted by muscle 150 during contractions, force buckle 156 was positioned around muscle 150, as shown in FIG. 11. In other examples, other types of sensors can also be used to determine muscle contraction parameters. For example, an accelerometer (e.g., a single axis accelerometer) can be attached to muscle 150 or EMG electrodes can be attached to muscle 150 to determine muscle movement parameters. In the example shown in FIG. 11, force sensor 158 included a three-axis accelerometer in addition to the load sensors.

Data was collected from force sensor 158 and force buckle 156 using a data acquisition system, and, in particular, the LabVIEW™ data acquisition system (National Instructions corporation of Austin, Tex.). A peak detection algorithm was employed in order to filter the data from force sensor 158 and force buckle 156 and obtain a single maximum force. Video data from cameras 152, 154 and force data from force buckle 156 and force sensor 158 were linked using the LabVIEW data acquisition system and MATLAB® (MathWorks, Inc. of Natick, Mass.) post processing system, which were both used to develop data acquisition and post processing features specifically for the study. In particular, the data from force buckle 158, force sensor 158, and cameras 152, 154 were automatically provided to a computing device, which was executing the LabVIEW™ data acquisition system and MATLAB® post processing system.

A set of data was obtained for each nonhuman subject and each muscle of interest for the respective nonhuman subject using a system similar to that shown in FIG. 11. The set of data included muscle parameters. In this study, the muscle parameters were muscle length, muscle thickness, muscle width, muscle volume, muscle mass, and a physiological cross-sectional area of the muscle. The set of data also included the in-line force indicated by force buckle 156, and the normal force indicated by force sensor 158 at the time the in-line force was determined. Various iterations of stimulation were performed on an ovine (sheep) subject in order to refine the type of muscle parameters selected for the data set, as well as to refine the surgical and data collection expedience and consistency. The data for each nonhuman subject was collected and analyzed using the Minitab statistical analysis software (Minitab, Inc. of State College, Pa.). Minitab was utilized to conduct a regression analysis for the collective sets of data obtained for the muscles of interest of each nonhuman subject. The regression equation resulting from the regression analysis was selected to be the transfer equation (Equation 1) based on the relatively low error rate of the regression equation in estimating the compressive force exerted by a muscle based on the in-line muscle force and muscle parameters.

FIG. 12 is a graph illustrating a plot of the normal force determined based on data from force sensor 158 versus the normal force determined based on data from force buckle 156 and cross-sectional area (CSA) volume of muscle 150. The graph illustrate in FIG. 12 was generated based on data from an ovine subject. An analysis of the variance resulting from the regression equation indicates that there is a standard error of less than about 1.3 pound force (about 5.78 Newtons), and a coefficient of determination (R²) of about 87.8%. The relatively low error demonstrates a relatively high accuracy of the transfer function for at least the ovine subject.

In order to confirm that the differences in geometric parameters of muscles for different sized nonhuman subjects did not substantially affect the transfer function, the various in-line force, normal force, and muscle parameters were determined for porcine and baboon subjects, in addition to the ovine subjects. The example transfer function (Equation 1) was determined based on the data from the different species of subjects. Thus, the variables of the transfer function may vary based on the specific data sets generated, which can vary on the number and type of muscles and subjects studied. The techniques described in this disclosure can be applied, however, to any number of subjects, data sets, and data from any number of different types of species. It is believed that the transfer function determined according to the techniques described in this disclosure and using the muscle parameters described in this disclosure will remain substantially similar and provide a relatively low error. An analysis of the variance resulting from the example regression equation (i.e., Equation 1) applied to the data from the ovine, porcine, and baboon subjects indicates that there is a standard error of less than about 2.3 pound force (about 10.23 Newtons), and a coefficient of determination (R²) of about 89.9%. The relatively low error demonstrates a relatively high accuracy of the transfer function and an ability to predict normal force levels across nonhuman subjects and different muscle groups of the nonhuman subjects using the transfer function.

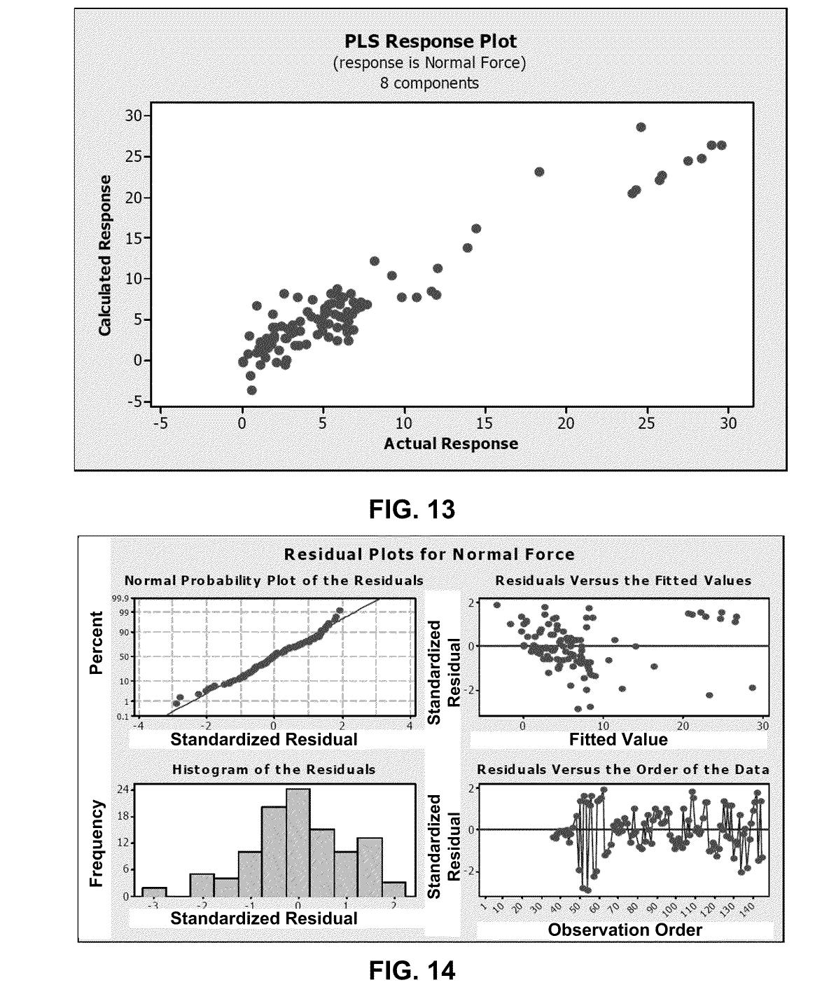

FIG. 13 is a partial least squares (PLS) plot generated using the regression data from sets of data obtained from the ovine, porcine, and baboon subjects. The partial least squares (PLS) plot shown in FIG. 13 demonstrates the high accuracy of the transfer function in identifying the relationship between the actual normal force determined via force sensor 158 at the time a particular in-line force was determined via force buckle 156 for a particular muscle 150 having particular parameters (e.g., muscle length, muscle width, muscle thickness, physiological cross-sectional area, muscle volume, and mass) and the normal force determined using the transfer function (Equation 1) and the known in-line force and muscle parameters.

An analysis of the variance resulting from the regression equation (transfer function, Equation 1) determined based on the sets of data obtained from the ovine, porcine, and baboon subjects indicates that there is a standard error of less than about 2.3 pound force, and a coefficient of determination (R²) of about 89.9%. The relatively low error demonstrates a relatively high accuracy of the transfer function and an ability to predict normal force levels across nonhuman subjects and different muscle groups of the nonhuman subjects using the transfer function. Statistical techniques, such as the Monte Carlo simulation methods, can be used to further decrease the standard error of the transfer function, which may further improve the ability to accurately predict a human normal muscle force using the transfer function.

FIG. 14 illustrates residual plots for the normal force. The residuals indicate a difference between the actual normal force determined via force sensor 158 (FIG. 11) and the regressed (fitted) function value of the normal force determined via the transfer function (Equation 1). The random scatter of the residuals indicates a high confidence in the regression equation (Equation 1).

It is believed that the skeletal muscles for the different species studied (i.e., ovine, porcine, and nonhuman primates) demonstrate relatively the same correlation between in-line force and normal force. Given the relatively consistent results between the different species, the studies conducted on the nonhuman subjects suggest that the transfer function that indicates the relationship between in-line muscle forces, muscle parameters (e.g., muscle length, muscle width, muscle thickness, physiological cross-sectional area, muscle volume, and mass), and transverse muscle forces is applicable to the human model. However, a human correlation model can be generated to further correlate the determined relationship between in-line muscle force and muscle parameters with normal muscle force.

A muscle may generate a particular in-line force and a resultant normal force for a particular motion. Therefore, the externally measured parameters used to determine the in-line force and, in some cases, the normal forces can be used to determine the cyclic quantities (e.g., occurrences) of particular patient motions (e.g., motion resulting an in-line or normal muscle force greater than predetermined threshold value or a particular type of patient motion). The cyclic quantities of these motions can be monitored over any suitable timeframe, such as, but not limited to, over a course of a day, multiple days, weeks or even months. The occurrences of muscle motion information may also be useful when designing an IMD and/or selecting an implant site for the IMD. For example, it may be desirable to implant an IMD at a tissue site in which certain patient motions do not cause the muscle proximate the IMD to contract, or a tissue site in which the IMD is exposed to infrequent muscle movement to limit the stresses exerted on the IMD. As another example of how the information regarding the frequency of particular muscle motion can be useful, it may be desirable to design an outer housing of the IMD to withstand the determined frequency of muscle movement, which can exert both compressive and shear stresses on the IMD housing.

In some examples, in order to determine the expected in vivo loading conditions for a particular patient, the in-line force generated by a target muscle or muscle group can be measured over a particular range of time (e.g., a day, a plurality of days, a week or a plurality of weeks). The in-line force can be used to determine the expected normal forces exerted by the target muscle or muscle group based on the transfer function (Equation 1), the variables of which may or may not be modified to be more specific to a human patient. The frequency of the normal forces, the magnitude of the normal forces, and other force parameters can then be referenced during design of the IMD, during selection of the type of IMD to implant in the patient (e.g., a particular model) or during selection of the IMD implant site.

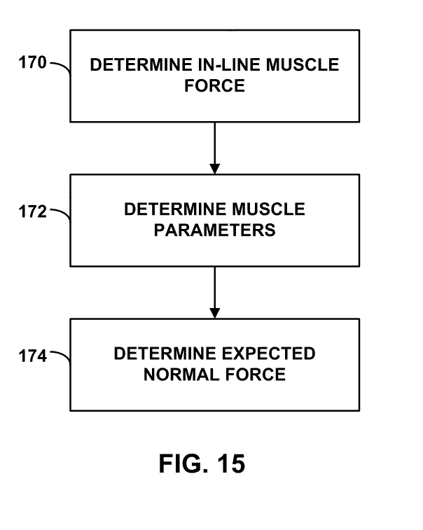

FIG. 15 is a flow diagram illustrating an example technique for determining an expected normal (or compressive) force exerted by a muscle or muscle group of interest (generally referred to in this disclosure as a target muscle). The technique shown in FIG. 15 is useful for determining the expected normal (or compressive) force exerted by a target muscle in a relatively noninvasive manner. In some examples, the expected normal (or compressive) force exerted by a target muscle can be determined without requiring surgery or at least as an outpatient procedure. While FIG. 15 is described with reference to processor 90 of programmer 24 (FIG. 6), in other examples, a processor of another device alone or with the assistance of a clinician can perform the technique shown in FIG. 15. In order to determine the normal force expected to be exerted by a target muscle,

According to the example technique, processor 90 determines the in-line force exerted by the target muscle (170). In some examples, processor 90 determines the in-line force (170) based on input received from a sensor, such as buckle sensor 156 (FIG. 11). In other examples, processor 90 determines the in-line force (170) based on the Hill Muscle Model. For example, processor 90 can receive an electrical signal from a sensor that indicates an EMG for the target muscle. Based on the EMG that indicates the electrical activation signal generated by a target muscle and a known length of the target muscle (which can be stored by memory 92 or inputted by a clinician via user interface 94), processor 90 can determine the in-line muscle force using the Hill Muscle Model. In yet another example, processor 90 can determine the in-line force (170) based on input provided by the clinician or another user via user interface 94 (FIG. 6).

Processor 90 can also determine muscle parameters (172). For example, processor 90 can retrieve stored muscle parameters from memory 92 or processor 90 can receive input from the clinician indicating the muscle parameters. In the example transfer function described above, the muscle parameters that are used to determine the expected compressive force includes muscle length, muscle width, muscle thickness, physiological cross-sectional area, muscle volume, and muscle mass. However, in other examples, processor 90 can determine other muscle parameters (e.g., other geometric parameters or other parameters indicating mass, such as density) in order to determine the normal force expected to be exerted by the target muscle.

After determining the in-line muscle force (170) and muscle parameters (172), processor 90 determines the expected normal force (174) using a transfer function that indicates a relationship between the normal force and the in-line muscle force and muscle parameters. An example of such a transfer function is provided above as Equation 1. However, as discussed above, the variables or other aspects of the transfer function may vary depending upon the data on which the transfer function is determined, where the data itself can change based on the number of subjects studied, the species of subjects studied, and the number and type of muscles studied. In general, the subjects are selected to be approximately the same size as humans or at least have muscles or muscle groups of interest that are the same size as human muscles.

In some examples, processor 90 can automatically determine at least one implant parameter for an IMD based on the compressive force. The implant parameter can be, for example, an implant site for an IMD, a type of IMD, or a design consideration of the IMD (e.g., a housing material, size, shape or other construction). For example, processor 90 can compare the compressive forces exerted by a plurality of muscles and generate a recommendation (e.g., presented via user interface 94 of programmer 24) based on the comparison (e.g., recommending the implant site proximate the muscle that is expected to exert the lowest compressive force). In other examples, processor 90 can determine the compressive force expected to be exerted by a muscle at a user-specified implant site and recommend a type of IMD to be implanted at the IMD. The IMD can be selected to be one that is capable of withstanding long-term exposure to the compressive forces without substantially affects on the IMD performance. The implant parameters (e.g., types of IMD and implant sites and associated muscles) can be stored by memory 92 of programmer 24 or a memory of another device. As another example of how processor 90 can select an implant parameter based on a determined compressive force, processor 90 can input the expected compressive forces for a plurality of muscles into a software program that designs the IMD outer housing.

The techniques described in this disclosure, including those attributed to IMD 16, IMD 40, IMD 50, IMD 60, programmer 24, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Many examples of the disclosure have been described. These and other examples are within the scope of the following claims. Various modifications may be made without departing from the scope of the following claims.

1. An implantable medical system comprising:
a housing;
a therapy delivery module substantially enclosed within the housing, wherein the therapy delivery module is configured to deliver therapy to a patient;
a component substantially enclosed within the housing; and
a mechanical stress sensor mechanically coupled to at least one of the housing or the component, wherein the mechanical stress sensor generates a signal indicative of a mechanical load exerted on the housing or the component.

2. The system of claim 1, wherein the component comprises a circuit board and the mechanical stress sensor is at least one of mechanically attached to the circuit board or integrated into the circuit board.

3. The system of claim 1, wherein the component comprises at least one of a processor or a power source of the therapy delivery module.

4. The system of claim 1, wherein the mechanical stress sensor is mechanically coupled to at least one of an interior surface or an exterior surface of the housing, or integrated with the interior surface or the exterior surface of the housing.

5. The system of claim 1, further comprising:
a telemetry module substantially enclosed within the housing; and
a processor substantially enclosed within the housing, wherein the processor is configured to receive the signal from the mechanical stress sensor and transmit information indicative of the signal to an external device via the telemetry module.

6. The system of claim 1, further comprising a processor that is configured to receive the signal from the mechanical stress sensor and automatically determine whether the mechanical load exerted on the housing is greater than or equal to a predetermined threshold based on the signal generated by the mechanical stress sensor.

7. The system of claim 6, wherein the processor is configured to generate an indication if the mechanical load exerted on the housing is greater than or equal to the predetermined threshold.

8. The system of claim 1, further comprising a processor that is configured to receive the signal from the mechanical stress sensor and automatically determine at least one parameter of the patient based on the signal.

9. The system of claim 8, wherein the processor is configured to control the therapy delivery module based on the determined parameter of the patient.

10. The system of claim 1, further comprising a processor and a memory, wherein the processor is configured to determine a mechanical stress signature based on the signal generated by the mechanical stress sensor and store the mechanical stress signature in the memory.

11. The system of claim 10, wherein the processor is configured to compare the mechanical stress signature to a predetermined signature template and generate an indication based on the comparison.

12. The system of claim 1, wherein the mechanical stress sensor comprises at least one of a printed circuit comprising pressure sensitive ink, a piezoresistor, a piezoelectric crystal, a load cell, a capacitive sensor, a pressure transducer, a force sensor, a displacement sensor or a strain gauge.

13. A method comprising:
implanting a mechanical stress indicator system in a patient, wherein the mechanical stress indicator system comprises:
a housing defining a form factor of an implantable medical device that delivers therapy to a patient;
a component enclosed within the housing; and
a mechanical stress sensor coupled to at least one of the housing or the component, wherein the mechanical stress sensor generates a signal indicative of a mechanical load exerted on the housing or the component; and
with a processor, determining an in vivo mechanical load exerted on the housing based on the signal generated by the mechanical stress sensor.

14. The method of claim 13, further comprising, with the processor, determining whether the mechanical load exerted on the housing is greater than or equal to a predetermined threshold based on the signal generated by the mechanical stress sensor.

15. The method of claim 13, further comprising, with the processor, determining at least one parameter of the patient based on the signal.

16. The method of claim 15, wherein the mechanical stress indicator system further comprises a therapy delivery module substantially enclosed within the housing, the method further comprising controlling therapy delivery by the therapy delivery module based on the determined parameter of the patient.

17. The method of claim 13, further comprising:
with the processor, determining a mechanical stress signature based on the signal generated by the mechanical stress sensor;
with the processor, comparing the mechanical stress signature to a predetermined signature template; and
with the processor, generating an indication based on the comparison.

18. A method comprising:
receiving a signal generated by a mechanical stress sensor implanted within a patient, wherein the signal is indicative of a mechanical load exerted on a housing that defines a form factor of an implantable medical device that delivers therapy to a patient, and wherein the mechanical stress sensor is coupled to at least one of the housing or a component enclosed within the housing;
with a processor, comparing at least one characteristic of the signal to a predetermined threshold value; and
with the processor, generating an indication based on the comparison.

19. The method of claim 18, wherein generating the indication comprises generating the indication if the at least one characteristic of the signal is greater than or equal to the predetermined threshold value.

20. The method of claim 18, further comprising, with the processor, transmitting the indication to a device separate from the mechanical stress sensor if the comparison of the at least one characteristic of the signal to the predetermined threshold value indicates the mechanical load exerted on the housing is greater than or equal to the predetermined threshold.
